(19) United States
(12) Patent Application Publication
(10) Pub. No.: US 2011/0243883 A1
(43) Pub. Date: Oct. 6, 2011

(54) POLYMERIC BIOLUBRICANTS FOR MEDICAL USE

(75) Inventors: Mark W. Grinstaff, Brookline, MA (US); Michel Wathier, Brookline, MA (US); Neel Joshi, Cambridge, MA (US); Stephanie Stoddart, Boston, MA (US)

(73) Assignee: TRUSTEES OF BOSTON UNIVERSITY, Boston, MA (US)

(21) Appl. No.: 13/119,526

(22) PCT Filed: Oct. 31, 2008
(86) PCT No.: PCT/US08/82125

§ 371 Date: Jun. 16, 2011

Related U.S. Application Data
(60) Provisional application No. 61/098,506, filed on Sep. 19, 2008.

(51) Int. Cl. A61K 47/34; C08G 69/48; A61K 38/18; A61K 38/39; A61K 31/22; A61K 31/80; A61K 31/755; A61K 33/42; A61K 31/728; A61P 19/02

(52) U.S. Cl. 424/78.31; 525/420; 525/54.1; 525/54.2; 514/772.1; 514/7.6; 514/17.2; 514/546; 424/78.37; 424/602; 514/54

(57) ABSTRACT

The present invention provides branched polymers which can be used as lubricants or shock absorbers in vivo. For example, the inventive polymers can be used as viscosupplements, viscoelastics, tissue space fillers, and/or anti-adhesive agents. Also provided are pharmaceutical compositions comprising the inventive polymers and methods of using them including, for example, in the treatment of arthritic and sport-injured knee joints; in reconstruction or cosmetic procedures, intervertebral disc repair, treatment of vocal cord problems, treatment of urinary incontinence, and prevention of adhesion formation following abdominal or gynecological surgery.

### CROSS REFERENCE TO RELATED APPLICATIONS

This application claims priority to U.S. Provisional Patent Application Ser. No. 61/098,506, filed Sep. 19, 2008, the entirety of which is hereby incorporated herein by reference.

### FIELD OF THE INVENTION

The present invention relates to polymeric biolubricants and uses thereof.

### BACKGROUND OF THE INVENTION

Osteoarthritis (OA), a non-inflammatory joint disease characterized by degeneration of joint cartilage, can affect one or more parts of the body, including hands and weight-bearing joints such as knees, hips, feet and the spine. When healthy, cartilage allows bones to glide over each other and has a shock absorber function. In osteoarthritis, the cartilage's surface layer breaks down and wears away, which allows the bones under the cartilage to rub together, causing the common OA symptoms of pain, swelling, and loss of motion of the joint. Furthermore, in joints such as the knees, osteoarthritis is often accompanied by loss of viscosity of the synovial fluid, a thick, gel-like substance that cushions the joint and provides lubrication to reduce friction of the bones.

Osteoarthritis is mainly associated with aging, with a prevalence of approximately 80% in individuals over 65. Despite being a condition that causes most problems to populations after retirement age, osteoarthritis is also rated the highest cause of work loss in the U.S. and Europe. In addition to age, risk factors known to be associated with osteoarthritis include obesity, traumatic injury and overuse due to sports and occupational stresses.

There is currently no cure for osteoarthritis, and available arthritis therapies are directed at the symptomatic relief of pain, and at improving, or at least maintaining, joint function. Generally, pain relievers such as non-steroidal anti-inflammatory drugs (NSAIDs) or COX-2 inhibitors are used, along with physical therapy. However, in the context of the recent withdrawals of COX-2 inhibitors, physicians are even more limited in their choice of treatment for osteoarthritis.

Viscosupplementation, a procedure involving the injection of gel-like substances (generally hyaluronates or called hyaluronic acid) into a joint to supplement the viscous properties of synovial fluid, has been shown to relieve pain in many osteoarthritis patients who do not get relief from analgesic drugs. The technique has been used in Europe and Asia for several years, but the U.S. Food and Drug Administration did not approve it until 1997. In current procedures of viscosupplementation, hyaluronate preparations are injected to replace or supplement the body's natural hyaluronan, a polysaccharide component of synovial fluid. The injections coat the articular cartilage surface, and thus provide a possible prophylactic barrier for the articular cartilage. However, due to their short lifetime within the joint (about a couple of days), hyaluronate preparations currently available have only limited long-term benefit to the patient and require injection of large quantities of the preparation and/or repeated injections.

### SUMMARY OF THE INVENTION

The present invention encompasses the recognition that there is a need for materials with improved performance for use in viscosupplementation for the treatment of osteoarthritis and other conditions affecting weight-bearing joints. In particular, materials with long lifetimes within injected biological fluids or tissues, such as joints, are highly desirable. In general, it is desirable that inventive polymers have protective characteristics comparable to synovial fluids.

Among other things, the present invention provides branched polymers which possess lubricating or shock absorbing properties and their use in joints. The inventive polymers, which can be viscous liquids or gels, are potential “bio-lubricants” that can find various applications in the biotechnology, pharmaceutical and medical fields. For example, the polymers described herein can be used in viscosupplementation (e.g., in the treatment of osteoarthritic or sport-injured knee joints). They can also be employed as viscoelastics used in cataract surgery, as fillers for cosmetic procedures or treatment of urinary incontinence, and as anti-adhesives for wound care.

More specifically, the present invention provides polymers having a branched chemical structure (e.g., without limitation dendrimers, hybrid linear-dendrimer and hyperbranched polymers).

In another aspect, the present invention provides pharmaceutical compositions comprising at least one pharmaceutically acceptable carrier and an effective amount of at least one inventive polymer described above.

In another aspect, the present invention provides methods of treating a diseased or injured synovial joint in a subject, such methods comprising injecting an effective amount of inventive polymer. In certain embodiments, injecting an effective amount of inventive polymer comprises performing a single injection. In other embodiments, injecting an effective amount of inventive polymer comprises performing at least two injections at different time points. Diseased or injured synovial joints that can be treated using these inventive methods include osteoarthritic joints and sport-injured joints, such as joints of the knee, hip, elbow, ankle, and wrist.

In another aspect, the present invention provides methods of repairing skin in a subject, such methods comprising administering to the subject an effective amount of inventive polymer. In certain embodiments, the polymer is injected to the area of skin to be repaired. In other embodiments, the polymer is topically applied to the area of skin to be repaired.

In still another aspect, the present invention provides methods of repairing an intervertebral disc in a subject, such methods comprising administering to the subject an effective amount of an inventive polymer. For example, the polymer may be injected to the intervertebral disc to be repaired.

In yet another aspect, the present invention provides methods of treating urinary incontinence in a subject, such methods comprising administering to the subject an effective amount of inventive polymer. The polymer may be injected to at least one defective area of the subject's urinary system.

In the methods of treatment of the invention, the polymer may be used as a viscous liquid or as a gel and may further comprise an additional substance, for example, a substance to be delivered to the area of administration of the polymer (e.g., joint, skin, intervertebral disc, urinary system). The additional substance may be one or more of a growth factor, a cytokine, a small molecule, an analgesic, an anesthetic, an antimicrobial agent, an antibacterial agent, an antiviral agent, an antifungal agent, an antibiotic, an anti-inflammatory agent, an antioxidant, and an antiseptic agent.

### BRIEF DESCRIPTION OF THE FIGURES

FIG. 1. Reaction of PAMAM (G3) with Methoxy-Polyethylene Glycol-Nitrophenyl Carbonate.

FIG. 2. NMR spectra of 2,000 molecular weight PEG acid taken in deuterated chloroform. As shown, the letters near each peak correspond to the hydrogen atoms of the same letter on the PEG acid molecule. The largest peak (B) relates to the hydrogen atoms on the repeating OCH₂CH₂ backbone, which consists of 44 units for 2,000 molecular weight PEG.

FIG. 3. NMR spectra of 5,000 molecular weight PEG acid taken in deuterated chloroform. The letters by each peak correspond to the hydrogen atoms of the same letter on molecule. The largest peak (B) relates to the hydrogen atoms on the repeating OCH₂CH₂ backbone, which consists of 122 units for 5,000 molecular weight PEG. The area of this peak is larger than that from 2,000 PEG acid since there 2.5 times as many repeating units.

FIG. 4. NMR spectrum of the 2,000 methoxy poly(ethylene glycol) polyamidoamine generation 2 (2K PEG-PAMAM G2) molecule taken in deuterated chloroform. Each of the different types of hydrogen atoms from each generation of PAMAM sum to yield one signal in the spectra. The hydrogen atoms bonded to the nitrogen atoms (D) are not shown because they are located downfield near a chemical shift of 8.0 ppm. Integrating peak C to 56 yields an integration of 24.9 for peak X. Since peak X ideally would have an integration of 32, the PEGylated dendrimer is 77.8% conjugated, resulting in a molecular weight of 30,556 g/mol

FIG. 5. NMR spectrum of the 5,000 methoxy poly(ethylene glycol)polyamidoamine generation 2 (5K PEG-PAMAM G2) molecule taken in deuterated chloroform. The hydrogen atoms bonded to the nitrogen atoms (D) are not shown because they are located downfield near a chemical shift of 8.0 ppm. Integrating peak C to 56 yields an integration of 29.5 for peak X. Since peak X ideally would have an integration of 32, the PEGylated dendrimer is 92.1% conjugated.

FIG. 6. NMR spectrum of the 2K PEG-PAMAM G3 molecule taken in deuterated chloroform. The hydrogen atoms bonded to the nitrogen atoms (D) are not shown because they are located downfield near a chemical shift of 8.0 ppm. Integrating peak C to 120 yields an integration of 63.8 for peak X, yielding 100% conjugation and resulting in a molecular weight of 74,109 g/mol.

FIG. 7. NMR spectrum of the 5K PEG-PAMAM G3 molecule taken in deuterated chloroform. The hydrogen atoms bonded to the nitrogen atoms (D) are not shown because they are located downfield near a chemical shift of 8.0 ppm. Integrating peak C to 120 yields an integration of 56.4 for peak X, yielding 88.1% conjugation and resulting in a molecular weight of 149,709 g/mol.

FIG. 8. Experimental setup for the cartilage-on-cartilage rheological testing. In a.) the three adapter pieces are attached to the rheometer whereas in b.) the cartilage plugs and lubricant are added to the setup.

FIG. 9. Generation zero (GO) linear-dendrimer hybrid

FIG. 10. Chemical structures of two lys-PEG hybride dendritic macromolecules synthesized.

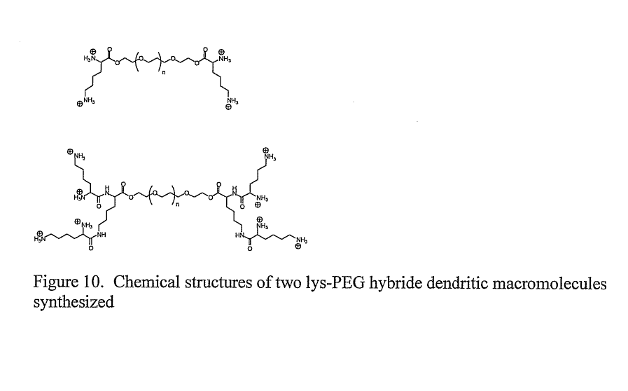

FIG. 11. Structures of various polymer embodiments for biolubrication.

### DEFINITIONS

Throughout the specification, several terms are employed that are defined in the following paragraphs.

The terms “individual” and “subject” are used herein interchangeably. They refer to a human or another mammal (e.g., primates, dogs, cats, goats, horses, pigs, mice, rabbits, and the like). In certain preferred embodiments, the subject is human. The terms do not denote a particular age, and thus encompass adults, children, and newborn.

The term “treatment” is used herein to characterize a method or process that is aimed at (1) delaying or preventing the onset of a disease or condition; (2) slowing down or stopping the progression, aggravation, or deterioration of the symptoms of the disease or condition; (3) bringing about ameliorations of the symptoms of the disease or condition; or (4) curing the disease or condition. A treatment may be administered prior to the onset of the disease, for a prophylactic or preventive action. Alternatively or additionally, the treatment may be administered after initiation of the disease or condition, for a therapeutic action.

The term “local”, when used herein to characterize the delivery, administration or application of a polymer of the present invention, or a pharmaceutical composition thereof, is meant to specify that the polymer or composition, is delivered, administered or applied directly to the site to be treated or in the vicinity of the site to be treated for a localized effect. For example, an inventive polymer used as a viscosupplement will generally be injected directly to an osteoarthritic knee joint; an inventive polymer used as tissue space filler will generally be injected directly to a diseased or damaged vocal cord, or to a skin area displaying lines or wrinkles. Preferably, local administration is effected without any significant absorption of components of the polymer into the patient's blood stream (to avoid a systemic effect).

A “pharmaceutical composition” is defined herein as comprising an effective amount of at least one active ingredient (e.g., an inventive polysaccharide mimic), and at least one pharmaceutically acceptable carrier.

As used herein, the term “pharmaceutically acceptable carrier” refers to a carrier medium which does not interfere with the effectiveness of the biological activity of the active ingredient(s) and which is not excessively toxic to the host at the concentration at which it is administered. The term includes solvents, dispersion media, coatings, antibacterial and antifungal agents, isotonic agents, absorption delaying agents, and the like. The use of such media and agents for pharmaceutically active substances is well known in the art (see for example, “Remington's Pharmaceutical Sciences”, E. W. Martin, ₁₈th Ed., 1990, Mack Publishing Co.: Easton, Pa., which is incorporated herein by reference in its entirety).

As used herein, the term “comparable to synovial fluid” is defined as falling within reasonable range of the values observed for synovial fluid such that similar functionalities or properties are observed. In instances herein, this term refers to lubricant properties of inventive polymers being comparable to lubricant properties of synovial fluids. Exemplary such properties include, but are not limited to, coefficient of friction, time of retention in a body cavity, tissue, or synovial space, biodegradability, and biocompatibility. Those of ordinary skill in the art would be aware of a variety of methods to assess whether the inventive polymers are comparable to synovial fluid as defined herein. In some embodiments, polymers for use in accordance with the present invention, show lubricant properties that vary by not more than 50% from measurements of the same property for synovial fluid; in some embodiments, polymers for use in accordance with the present invention show lubricant properties that vary by not more than 40%, 30%, 20%, 15%, 10%, 9%, 8%, 7%, 6%, 5% or less from from measurements of the same property for synovial fluid. In some embodiments, polymers for use in accordance with the present invention show lubricant properties that differ from synovial fluid by not more than a factor of 2, 3, 4, 5, 6, 7, 8, 9, 10, 15, 20, 30, 40, or 50. In some embodiments, polymers for use in accordance with the present invention, show time of retention in a synovial space that is similar to HA or can be as short as 2 days and as long as 2 years.

As used herein, the term “effective amount” refers to any amount of a molecule, compound or composition that is sufficient to fulfill its intended purpose(s), i.e., to elicit a desired biological or medicinal response in a tissue or subject. Examples of intended purposes of an inventive polymer include, but are not limited to, to provide viscosupplementation to a joint, to allow soft tissue augmentation, to prevent or reduce adhesion formation, to facilitate tissue manipulation, and/or to maintain, support or protect soft tissue. Those of ordinary skill in the art would be aware of a variety of methods to assess the amount comprising an effective amount as defined herein.

As used herein, the term “soft tissue augmentation” includes, but is not limited to, dermal tissue augmentation; filling of lines, folds, wrinkles, minor facial depressions, cleft lips and the like, especially in the face and neck; correction of minor deformities due to aging, disease, including in the hands and feet, fingers and toes; augmentation of the vocal cords or glottis to rehabilitate speech; dermal filling of sleep lines and expression lines; replacement of dermal and subcutaneous tissue lost due to aging; lip augmentation; filling of crow's feet and the orbital groove around the eye; breast augmentation; chin augmentation; augmentation of the cheek and/or nose; filling of indentations in the soft tissue, dermal or subcutaneous, due to, e.g., overzealous liposuction or other trauma; filling of acne or traumatic scars and rhytids; filling of nasolabial lines, nasoglobellar lines and infraoral lines.

As used herein, the term “soft tissue” includes all tissue of the body except bone. Examples of soft tissue include, but are not limited to, muscles, tendons, fibrous tissues, fat, blood vessels, nerves, and synovial tissues.

The terms “bioactive agent” and “biologically active agent” are used herein interchangeably. They refer to compounds or entities that alter, inhibit, activate or otherwise affect biological or chemical events. For example, bioactive agents may include, but are not limited to, vitamins, anti-cancer substances, antibiotics, immunosuppressants, anti-viral substances, enzyme inhibitors, opioids, hypnotics, lubricants, tranquilizers, anti-convulsants, muscle relaxants, anti-spasmodics and muscle contractants, anti-glaucoma compounds, modulators of cell-extracellular matrix interactions including cell growth inhibitors and anti-adhesion molecules, vasodilating agents, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, imaging agents. A more complete, although not exhaustive, listing of classes and specific drugs suitable for use in the present invention may be found in “Pharmaceutical Substances: Synthesis, Patents, Applications” by A. Kleeman and J. Engel, Thieme Medical Publishing, 1999; and the “Merck Index: An Encyclopedia of Chemicals, Drugs, and Biologicals”, S. Budavari et al. (Eds), CRC Press, 1996, both of which are incorporated herein by reference.

The term “small molecule” refers to molecules, whether naturally-occurring or artificially created (e.g., via chemical synthesis) that have a relatively low molecular weight. In some embodiments, small molecules are biologically active in that they produce a local or systemic effect in animals. In some embodiments, small molecules are biologically active in that they produce a local or systemic effect in mammals. In some embodiments, small molecules are biologically active in that they produce a local or systemic effect in humans. Typically, small molecules have a molecular weight of less than about 1,500 Da. In certain embodiments, the small molecule is a drug. In certain embodiments, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered suitable for use with the present polymers.

The terms “polysaccharide”, “carbohydrate”, and “oligosaccharide” are used herein interchangeably. They refer to a compound that comprises at least two sugar units, or derivatives thereof. Polysaccharides may be purified from natural sources such as plants or may be synthesized de novo in the laboratory. Polysaccharides isolated from natural sources may be modified chemically to change their chemical or physical properties (e.g., reduced, oxidized, phosphorylated, crosslinked). Carbohydrate polymers or oligomers may include natural sugars (e.g., glucose, fructose, galactose, mannose, arabinose, ribose, xylose, etc.) and/or modified sugars (e.g., 2′-fluororibose, 2′-deoxyribose, etc.). Polysaccharides may also be either straight or branched. They may contain both natural and/or unnatural carbohydrate residues. The linkage between the residues may be the typical ether linkage found in nature or may be a linkage only available to synthetic chemists. Examples of polysaccharides include cellulose, maltin, maltose, starch, modified starch, dextran, poly(dextrose), and fructose. Glycosaminoglycans are also considered polysaccharides. Sugar alcohol, as used herein, refers to any polyol such as sorbitol, mannitol, xylitol, galactitol, erythritol, inositol, ribitol, dulcitol, adonitol, arabitol, dithioerythritol, dithiothreitol, glycerol, isomalt, and hydrogenated starch hydrolysates.

An entity is herein said to be “associated with” another entity if they are linked by a direct or indirect, covalent or non-covalent interaction. In certain embodiments, the association is covalent. Desirable non-covalent interactions include hydrogen bonding, van der Walls interactions, hydrophobic interactions, magnetic interactions, electrostatic interactions, or combinations thereof.

In general, the term “aliphatic”, as used herein, includes both saturated and unsaturated, straight chain (i.e., unbranched) or branched aliphatic hydrocarbons, which are optionally substituted with one or more functional groups, as defined below. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having 1-6 carbon atoms. In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms.

Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents, as previously defined. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl(propargyl), 1-propynyl and the like.

The term “alicyclic”, as used herein, refers to compounds which combine the properties of aliphatic and cyclic compounds and include but are not limited to cyclic, or polycyclic aliphatic hydrocarbons and bridged cycloalkyl compounds, which are optionally substituted with one or more functional groups, as defined below. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl-n, cyclohexyl, —CH₂-cyclohexyl, cyclohexenylethyl, cyclohexanylethyl, norborbyl moieties and the like, which again, may bear one or more substituents.

The term “heteroaliphatic”, as used herein, refers to aliphatic moieties in which one or more carbon atoms in the main chain have been substituted with an heteroatom. Thus, a heteroaliphatic group refers to an aliphatic chain which contains one or more oxygen sulfur, nitrogen, phosphorus or silicon atoms, e.g., in place of carbon atoms. Heteroaliphatic moieties may be saturated or unsaturated, branched or linear (i.e., unbranched), and substituted or unsubstituted. Substituents include, but are not limited to, any of the substituents mentioned below, i.e., the substituents recited below resulting in the formation of a stable compound.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)N(R^{o})₂; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}²; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₈ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or polycyclic ring having 0-4 heteroatoms independently selected from nitrogen, oxygen, or sulfur, which may be substituted as defined below.

The term “heteroalicyclic”, as used herein, refers to compounds which combine the properties of heteroaliphatic and the cyclic compounds and include but are not limited to saturated and unsaturated mono- or polycyclic heterocycles such as morpholino, pyrrolidinyl, furanyl, thiofuranyl, pyrrolyl, etc, which are optionally substituted with one or more functional groups. Substituents include, but are not limited to, any of the substituents mentioned below, i.e., the substituents recited below resulting in the formation of a stable compound.

The term “alkyl”, as used herein, refers to saturated, straight- or branched-chain hydrocarbon radicals derived from a hydrocarbon moiety containing between one and twenty carbon atoms by removal of a single hydrogen atom, which alkyl groups are optionally substituted with one or more functional groups. Substituents include, but are not limited to, any of the substituents mentioned below, i.e., the substituents recited below resulting in the formation of a stable compound. Examples of alkyl radicals include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The term “alkoxy”, as used herein, refers to an alkyl group, as previously defined, attached to the parent molecular moiety through an oxygen atom. Examples include, but are not limited to, methoxy, ethoxy, propoxy, isopropoxy, n-butoxy, tert-butoxy, neopentoxy, and n-hexoxy.

The term “alkenyl” denotes a monovalent group derived from a hydrocarbon moiety having at least one carbon-carbon double bond, which alkenyl group is optionally is substituted with one or more functional groups. In certain embodiments, an alkenyl group contains between one and twenty carbon atoms. Substituents include, but are not limited to, any of the substituents mentioned below, i.e., the substituents recited below resulting in the formation of a stable compound. Alkenyl groups include, for example, ethenyl, propenyl, butenyl, 1-methyl-2-buten-1-yl, and the like.

The term “alkynyl”, as used herein, refers to a monovalent group derived from a hydrocarbon having at least one carbon-carbon triple bond, which alkynyl group is optionally substituted. In certain embodiments, an alkynyl group contains between one and twenty carbon atoms. Substituents include, but are not limited to, any of the substituents mentioned below, i.e., the substituents recited below resulting in the formation of a stable compound. Representative alkynyl groups include ethynyl, 2-propynyl(propargyl), 1-propynyl, and the like.

The term “amine”, as used herein, refers to one, two, or three alkyl groups, as previously defined, attached to the parent molecular moiety through a nitrogen atom. The term “alkylamino” refers to a group having the structure —NHR′ wherein R′ is an alkyl group, as previously defined; and the term “dialkylamino” refers to a group having the structure —NR′R″, wherein R′ and R″ are each independently selected from the group consisting of alkyl groups. The term “trialkylamino” refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “aryl”, as used herein, refers to stable mono- or polycyclic, unsaturated moieties having preferably 3-14 carbon atoms, each of which may be substituted or unsubstituted. Substituents include, but are not limited to, any of the substituents mentioned below, i.e., the substituents recited below resulting in the formation of a stable compound. The term aryl may refer to a mono- or bicyclic carbocyclic ring system having one or two aromatic rings including, but not limited to, phenyl, naphthyl, tetrahydronaphthyl, indanyl, indenyl and the like.

The term “heteroaryl”, as used herein refers to a stable heterocyclic or polyheterocyclic, unsaturated radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms. Heteroaryl moieties may be substituted or unsubstituted. Substituents include, but are not limited to, any of the substituents mentioned below, i.e., the substituents recited below resulting in the formation of a stable compound. Examples of heteroaryl nuclei include pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will also be appreciated that aryl and heteroaryl moieties, as defined herein, may be attached via an aliphatic, alicyclic, heteroaliphatic, heteroalicyclic, alkyl or heteroalkyl moiety and thus also include -(aliphatic)aryl, -(heteroaliphatic)aryl, -(aliphatic)heteroaryl, -(heteroa-liphatic)heteroaryl, -(alkyl)aryl, -(heteroalkyl)aryl, -(heteroalkyl)aryl, and -(heteroalkyl)-heteroaryl moieties. Thus, as used herein, the phrases “aryl or heteroaryl” and “aryl, heteroaryl, -(aliphatic)aryl, -(heteroaliphatic)aryl, -(aliphatic)heteroaryl, -(heteroaliphatic)heteroaryl, -(alkyl)aryl, -(heteroalkyl)aryl, -(heteroalkyl)aryl, and -(heteroalkyl)heteroaryl” are interchangeable.

The term “carboxylic acid”, as used herein, refers to a group of formula —CO₂H.

The terms “halo”, “halide”, and “halogen”, as used herein, refers to an atom selected from fluorine, chlorine, bromine, and iodine.

The term “methylol”, as used herein, refers to an alcohol group of structure —CH₂OH.

The term “hydroxyalkyl” refers to an alkyl group, as defined above, bearing at least one OH group.

The term “mercaptoalkyl”, a used herein, refers to an alkyl group, as defined above, bearing at least one SH group.

The term “heterocyclic”, as used herein, refers to a non-aromatic partially unsaturated or fully saturated 3- to 10-membered ring system, which includes single rings of 3 to 8 atoms in size and bi- and tri-cyclic ring systems which may include aromatic six-membered aryl or aromatic heterocyclic groups fused to a non-aromatic ring. Heterocyclic moieties may be substituted or unsubstituted. Substituents include, but are not limited to, any of the substituents mentioned below, i.e., the substituents recited below resulting in the formation of a stable compound. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized.

The term “acyl”, as used herein, refers to a group comprising a carbonyl group of the formula C═O. Examples of acyl groups include aldehydes, ketones, carboxylic acids, acyl halides, anhydrides, thioesters, amides, urea, carbamate, and carboxylic esters.

The term “hydrocarbon”, as used herein, refers to any chemical group comprising hydrogen and carbon. The hydrocarbon may be substituted or unsubstituted. The hydrocarbon may be unsaturated, saturated, branched, unbranched, cyclic, polycyclic, or heterocyclic. Illustrative hydrocarbons include, for example, methyl, ethyl, n-propyl, iso-propyl, cyclopropyl, allyl, vinyl, n-butyl, tert-butyl, ethynyl, cyclohexyl, methoxy, diethylamino, and the like. As would be known to one skilled in this art, all valencies must be satisfied in making any substitutions. Likewise a fluorocarbon as used herein refers to any chemical group comprising more fluorine than hydrogen with carbon. hydrocarbon may be substituted or unsubstituted. The fluorocarbon may be unsaturated, saturated, branched, unbranched, cyclic, polycyclic, or heterocyclic.

The term “substituted”, whether preceded by the term “optionally” or not, refers to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Heteroatoms such as nitrogen may have hydrogen substituents and/or any permissible substituents of organic compounds described herein which satisfy the valencies of the heteroatoms. Examples of substituents include, but are not limited to aliphatic; alicyclic; heteroaliphatic; heteroalicyclic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —NCO —CF₃; —CH₂CF₃; —CHCl₂; —CH₂ORₓ; —CH₂CH₂ORₓ; —CH₂N(Rₓ)₂; —CH₂SO₂CH₃; —C(O)Rₓ; —CO₂(Rₓ); —CON(Rₓ)₂; —OC(O)Rₓ; —C(O)OC(O)Rₓ; —OCO₂Rₓ; —OCON(Rₓ)₂; —N(Rₓ)₂; —S(O)₂Rₓ; —NRₓ(CO)Rₓ, —NRₓ(CO)N(Rₓ)₂ wherein each occurrence of Rₓ independently includes, but is not limited to, H, aliphatic, alicyclic, heteroaliphatic, heteroalicyclic, aryl, heteroaryl, alkylaryl, or alkylheteroaryl, wherein any of the aliphatic, alicyclic, heteroaliphatic, heteroalicyclic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted.

Dendritic polymers are globular monodispersed or nearly monodispersed polymers composed of repeated branching units emitting from a central core. (U.S. Pat. No. 5,714,166; U.S. Pat. No. 4,289,872; U.S. Pat. No. 4,435,548; U.S. Pat. No. 5,041,516; U.S. Pat. No. 5,362,843; U.S. Pat. No. 5,154,853; U.S. Pat. No. 05,739,256; U.S. Pat. No. 5,602,226; U.S. Pat. No. 5,514,764; Bosman, A. W.; Janssen, H. M.; Meijer, E. W. Chem. Rev. 1999, 99, 1665-1688. Fischer, M.; Vogtle, F. Angew. Chem. Int. Ed. 1999, 38, 884-905. Zeng, F.; Zimmerman, S. C. Chem. Rev. 1997, 97, 1681-1712. Tomalia, D. A.; Naylor, A. M.; Goddard, W. A. Angew. Chem. Int. Ed. Engl. 1990, 29, 138.) These macromolecules are synthesized using either a divergent (from core to surface) (Buhleier, W.; Wehner, F. V.; Vogtle, F. Synthesis 1987, 155-158. Tomalia, D. A.; Baker, H.; Dewald, J.; Hall, M.; Kallos, G.; Martin, S.; Roeck, J.; Ryder, J.; Smith, P. Polymer Journal 1985, 17, 117-132. Tomalia, D. A.; Baker, H.; Dewald, J.; Hall, M.; Kallos, G.; Martin, S.; Roeck, J.; Ryder, J.; Smith, P. Macromolecules 1986, 19, 2466. Newkome, G. R.; Yao, Z.; Baker, G. R.; Gupta, V. K. J. Org. Chem. 1985, 50, 2003.)¹ or a convergent (from surface to core) (Hawker, C. J.; Frechet, J. M. J. J. Am. Chem. Soc. 1990, 112, 7638-7647) approach. This research area has undergone tremendous growth in the last decade since the early work of Tomalia and Newkome. Compared to linear polymers, dendrimers are highly ordered, possess high surface area to volume ratios, and exhibit numerous end groups for functionalization. Consequently, dendrimers display several favorable physical properties for both industrial and biomedical applications including: small polydispersity indexes (PDI), low viscosities, high solubility and miscibility, and excellent adhesive properties. The majority of dendrimers investigated for biomedical/biotechnology applications (e.g., MRI, gene delivery, and cancer treatment) are derivatives of aromatic polyether or aliphatic amides and thus are not ideal for in vivo uses. (Service, R. F. Science 1995, 267, 458-459. Lindhorst, T. K.; Kieburg, C. Angew. Chem. Int. Ed. 1996, 35, 1953-1956. Ashton, P. R.; Boyd, S. E.; Brown, C. L.; Yayaraman, N.; Stoddart, J. F. Angew. Chem. Int. Ed. 1997, 1997, 732-735. Wiener, E. C.; Brechbeil, M. W.; Brothers, H.; Magin, R. L.; Gansow, O. A.; Tomalia, D. A.; Lauterbur, P. C. Magn. Reson. Med. 1994, 31, 1-8. Wiener, E. C.; Auteri, F. P.; Chen, J. W.; Brechbeil, M. W.; Gansow, O. A.; Schneider, D. S.; Beldford, R. L.; Clarkson, R. B.; Lauterbur, P. C. J. Am. Chem. Soc. 1996, 118, 7774-7782. Toth, E.; Pubanz, D.; Vauthey, S.; Helm, L.; Merbach, A. E. Chem. Eur. J. 1996, 2, 1607-1615. Adam, G. A.; Neuerburg, J.; Spuntrup, E.; Muhl;er, A.; Scherer, K.; Gunther, R. W. J. Magn. Reson. Imag. 1994, 4, 462-466. Bourne, M. W.; Margerun, L.; Hylton, N.; Campion, B.; Lai, J. J.; Dereugin, N.; Higgins, C. B. J. Magn. Reson. Imag. 1996, 6, 305-310. Miller, A. D. Angew. Chem. Int. Ed. 1998, 37, 1768-1785. Kukowska-Latallo, J. F.; Bielinska, A. U.; Johnson, J.; Spinder, R.; Tomalia, D. A.; Baker, J. R. Proc. Natl. Acad. Sci. 1996, 93, 4897-4902. Hawthorne, M. F. Angew. Chem. Int. Ed. 1993, 32, 950-984. Qualmann, B.; Kessels M. M.; Musiol H.; Sierralta W. D.; Jungblut P. W.; L., M. Angew. Chem. Int. Ed. 1996, 35, 909-911). Each patent and publication cited above and hereinafter is expressly incorporated into the subject application as if set forth fully therein.

### DETAILED DESCRIPTION OF CERTAIN EMBODIMENTS

As described herein, the present invention provides, among other things, branched polymers (e.g., without limitation dendrimers, hybrid linear-dendrimer and hyperbranched polymers) that are useful as biolubricants, for instance, in vivo for the treatment of OA. Inventive polymers, which can be viscous liquids or gels, can find various applications in the biotechnology, pharmaceutical and medical fields. In some embodiments, the present invention is characterized by having biolubricant properties comparable to those of synovial fluid. Such properties include, but are not limited to, coefficient of friction, biodegradability, biocompatibility, and good retention in a body cavity, tissue, or synovial space. In various embodiments, inventive polymers have molecular weights greater than 5000 g/mol. In some embodiments, the molecular weight ranges from 5000 g/mol to 10,000,000 g/mol. In some embodiments, the molecular weight ranges from 5000 g/mol to 5,000,000 g/mol. In some embodiments, the molecular weight ranges from 10,000 g/mol to 1,000,000 g/mol. In some embodiments, the molecular weight ranges from 10,000 g/mol to 500,000 g/mol. In some embodiments, the molecular weight ranges from 20,000 g/mol to 400,000 g/mol. In some embodiments, the molecular weight ranges from 20,000 g/mol to 250,000 g/mol. In some embodiments, the molecular weight ranges from 20,000 g/mol to 150,000 g/mol.

### I. Polymers

The present invention provides noncrosslinkable or crosslinkable branched polymers (including copolymers). In certain embodiments, these polymers are selected from the group consisting of dendrimers, hybrid linear-dendrimers, or hyperbranched polymers according to one of the general formulas below:

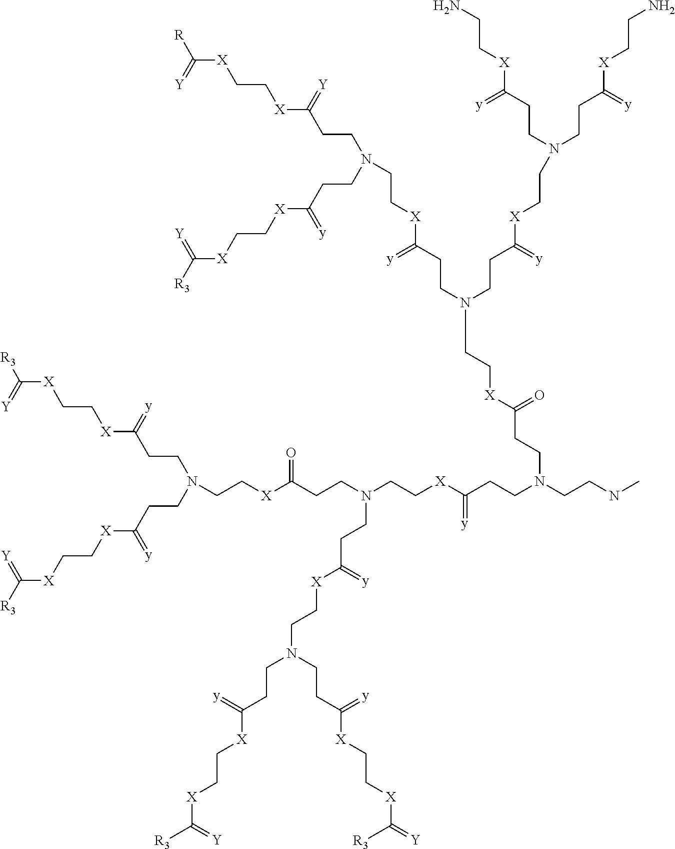

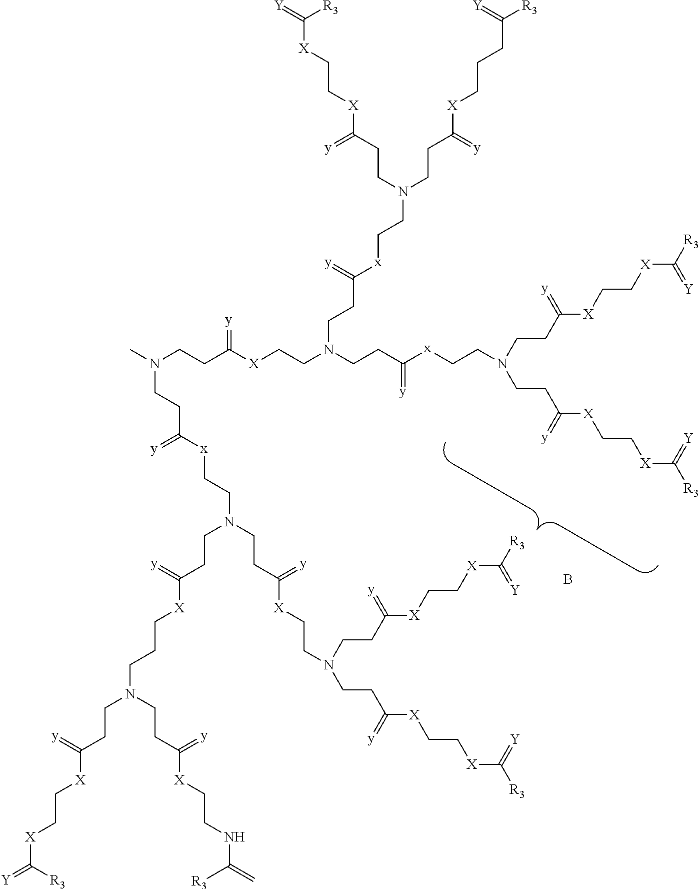

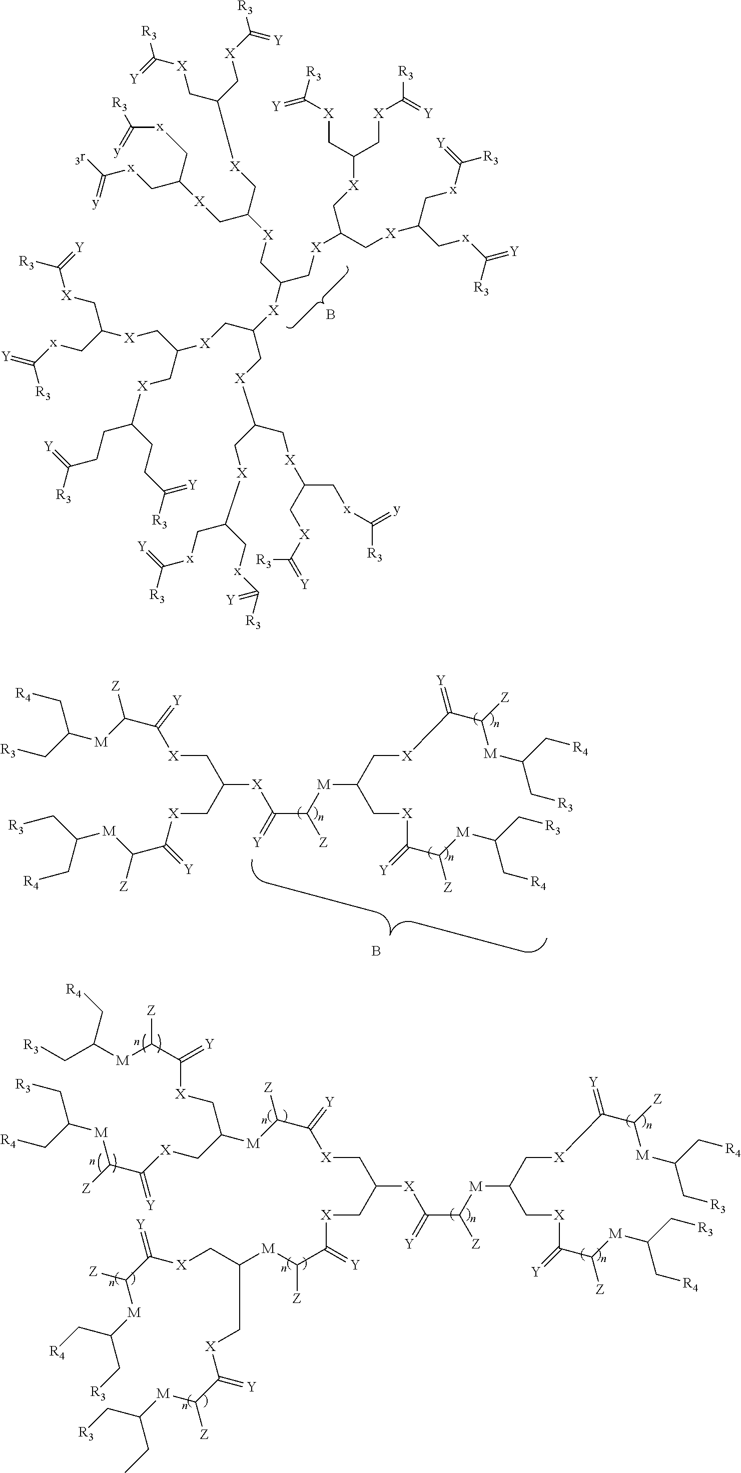

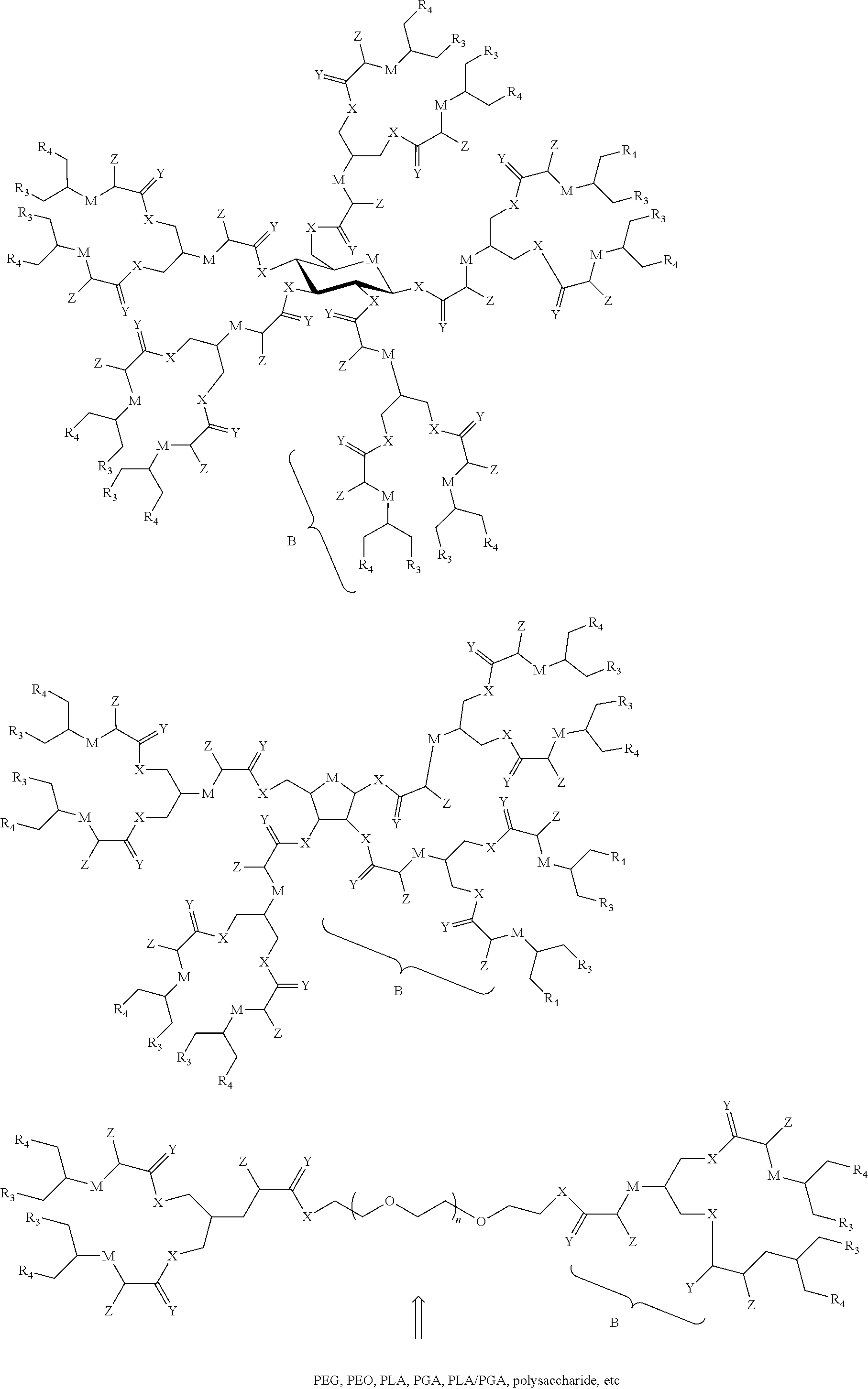

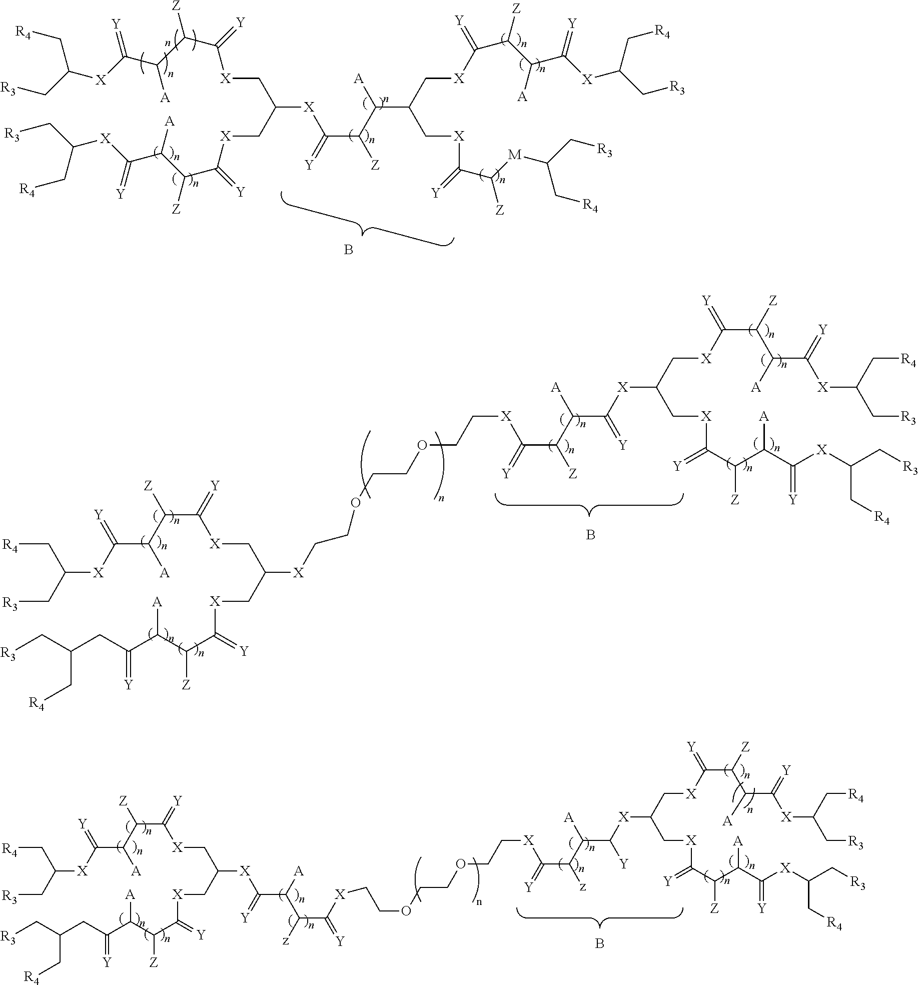

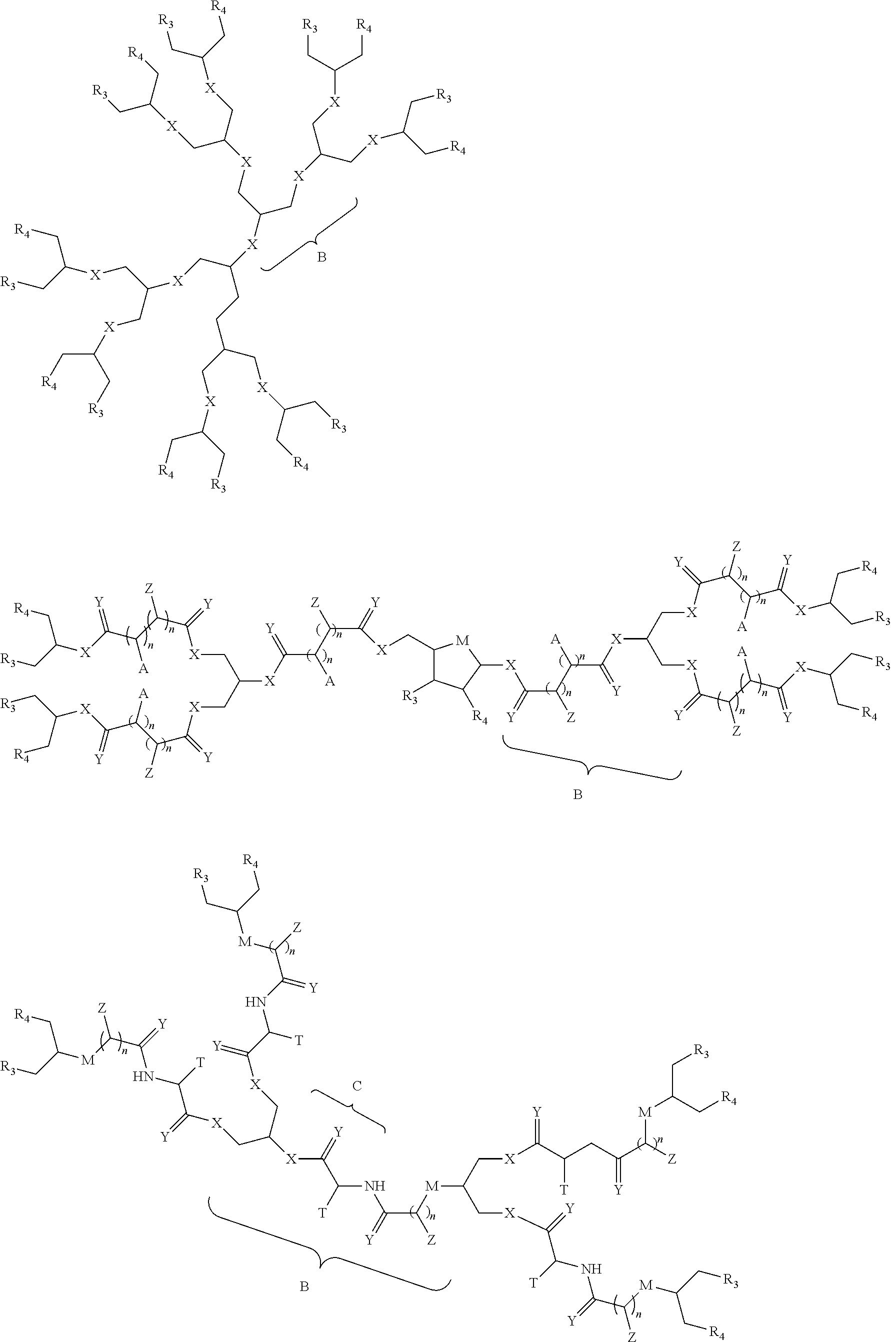

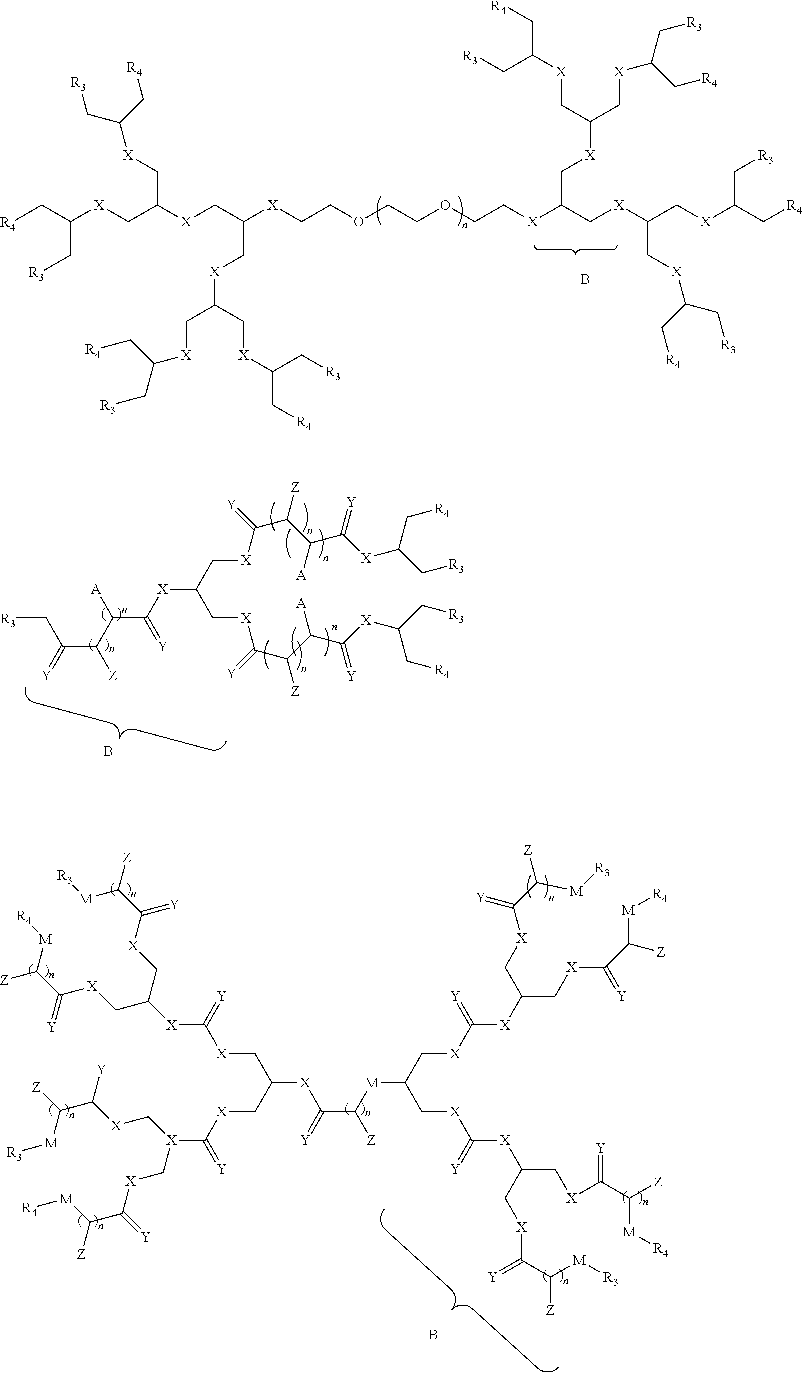

wherein:
n is an integer independently selected from 0 to 50, inclusive;
c is a natural or un-natural amino acid;
each occurrence of R₃, R₄, A, and Z is independently selected from the group consisting of a repeat pattern of B, an optionally substituted C₁₋₅₀ aliphatic group, —H, —OH, —CH₃, carboxylic acid, sulfate, phosphate, aldehyde, methoxy, amine, amide, thiol, disulfide, straight or branched chain alkane, straight or branched chain alkene, straight or branched chain ester, straight or branched chain ether, straight or branched chain silane, straight or branched chain urethane, straight or branched chain carbonate, straight or branched chain sulfate, straight or branched chain phosphate, straight or branched chain thiol urethane, straight or branched chain amine, straight or branched chain thiol urea, straight or branched chain thiol ether, straight or branched chain thiol ester, a carboxylic acid protecting group, and a linker moiety; and
each occurrence of X, Y, and M is independently selected from the group consisting of O, S, Se or any other isoelectronic species of oxygen; and or N(R′)_{n′}, wherein R′ is hydrogen or an optionally substituted C₁₋₂₀ aliphatic group; and wherein n′ is an integer from 1-4, inclusive.

The polymer having a straight or branched chain of 1-50 carbon atoms and wherein the chain is fully saturated, fully unsaturated, and any combination therein.

The polymer wherein straight or branched chains are the same number of carbons or different and wherein R₃, R₄, A, Z are any combination of linkers selected from the group consisting of methylenes, esters, silanes, ureas, amides, amines, urethanes, thiol-urethanes, carbonates, thio-ethers, thio-esters, sulfates, phosphates and ethers.

The polymer wherein chains include at least one selected from hydrocarbons, flourocarbons, halocarbons, alkenes, and alkynes.

The polymer wherein said chains include polyethers, polyesters, polyamines, polyacrylic acids, polyamino acids, polynucleic acids and polysaccharides of molecular weight ranging from 200-1,000,000, and wherein said chain contains 1 or more photopolymerizable group.

The polymer wherein the chains include at least one of PPG, PEG, PLA, PGA, PGLA, and PMMA or various molecular weights from 500 to 50,000 g/mol is attached.

A block or random copolymer which includes at least one terminal group selected from the group consisting of amines, thiols, amides, phosphates, sulphates, hydroxides, alkenes, and alkynes.

The polymer wherein an amino acid is attached to Z, A, R₃, and/or R₄.

The polymer wherein a polypeptide is attached to Z, A, R₃, and/or R₄.

The polymer wherein an antibody is attached to Z, A, R₃, and/or R₄.

The polymer wherein a nucleotide is attached to Z, A, R₃, and/or R₄.

The polymer wherein a nucleoside is attached to Z, A, R₃, and/or R₄.

The polymer wherein an oligonucleotide is attached to Z, A, R₃, and/or R₄.

The polymer wherein a ligand is attached to Z, A, R₃, and/or R₄ that binds to a biological receptor.

The polymer wherein a pharmaceutical agent is attached to Z, A, R₃, and/or R₄.

The polymer wherein a carbohydrate is attached to Z, A, R₃, and/or R₄.

The polymer of wherein a PET or MRI contrast agent is attached to Z, A, R₃, and/or R₄.

The polymer wherein the contrast agent is Gd(DPTA).

The polymer wherein an iodated compound for X-ray imagaging is attached to Z, A, R₃, and/or R₄.

The polymer wherein a pharmaceutical agent is attached to Z, A, R₃, and/or R₄ and is at least one selected from the group consisting of antibacterial, anticancer, anti-inflammatory, and antiviral.

The polymer wherein the carbohydrate is mannose or sialic acid.

In some embodiments, provided polymers are characterized by having characteristics comparable to those of synovial fluid.

In some embodiments, inventive polymers have molecular weights greater than 5000 g/mol (e.g., as determined by NMR). In some embodiments, the molecular weight ranges from 5000 g/mol to 10,000,000 g/mol. In some embodiments, the molecular weight ranges from 5000 g/mol to 9,000,000 g/mol. In some embodiments, the molecular weight ranges from 5000 g/mol to 8,000,000 g/mol. In some embodiments, the molecular weight ranges from 5000 g/mol to 7,000,000 g/mol. In some embodiments, the molecular weight ranges from 5000 g/mol to 6,000,000 g/mol. In some embodiments, the molecular weight ranges from 5000 g/mol to 5,000,000 g/mol. In some embodiments, the molecular weight ranges from 5000 g/mol to 4,000,000 g/mol. In some embodiments, the molecular weight ranges from 5000 g/mol to 3,000,000 g/mol. In some embodiments, the molecular weight ranges from 5000 g/mol to 2,000,000 g/mol. In some embodiments, the molecular weight ranges from 5000 g/mol to 1,000,000 g/mol. In some embodiments, the molecular weight ranges from 10,000 g/mol to 1,000,000 g/mol. In some embodiments, the molecular weight ranges from 10,000 g/mol to 900,000 g/mol. In some embodiments, the molecular weight ranges from 10,000 g/mol to 800,000 g/mol. In some embodiments, the molecular weight ranges from 10,000 g/mol to 700,000 g/mol. In some embodiments, the molecular weight ranges from 10,000 g/mol to 600,000 g/mol. In some embodiments, the molecular weight ranges from 10,000 g/mol to 500,000 g/mol. In some embodiments, the molecular weight ranges from 10,000 g/mol to 400,000 g/mol. In some embodiments, the molecular weight ranges from 10,000 g/mol to 300,000 g/mol. In some embodiments, the molecular weight ranges from 20,000 g/mol to 400,000 g/mol. In some embodiments, the molecular weight ranges from 20,000 g/mol to 300,000 g/mol. In some embodiments, the molecular weight ranges from 20,000 g/mol to 250,000 g/mol. In some embodiments, the molecular weight ranges from 20,000 g/mol to 200,000 g/mol. In some embodiments, the molecular weight ranges from 20,000 g/mol to 150,000 g/mol. In some embodiments, the molecular weight ranges from 30,000 g/mol to 250,000 g/mol. In some embodiments, the molecular weight ranges from 40,000 g/mol to 250,000 g/mol. In some embodiments, the molecular weight ranges from 40,000 g/mol to 225,000 g/mol. In some embodiments, the molecular weight ranges from 40,000 g/mol to 200,000 g/mol. In some embodiments, the molecular weight ranges from 40,000 g/mol to 180,000 g/mol. In some embodiments, the molecular weight ranges from 50,000 g/mol to 180,000 g/mol. In certain embodiments, inventive polymers have a molecular weight of approximately 30,000-40,000 g/mol. In certain embodiments, inventive polymers have a molecular weight of approximately 75,000-90,000 g/mol. In certain embodiments, inventive polymers have a molecular weight of approximately 140,000-180,000 g/mol. In some embodiments, inventive polymers have a large molecular weight to increase retention time at the site of administration. A large molecular weight is defined as a molecular weight between 3,000,000 g/mol and 10,000,000 g/mol (and any sub-range between these two endpoints). Methods of NMR analysis used to approximate molecular weights of the inventive polymers are known to those of ordinary skill in the art.

In some embodiments, the extent of conjugation of terminal amine groups of the inventive polymers with PEG ranges from 1-100% as measured by NMR analysis. In some embodiments, the extent of conjugation ranges from 25-100%. In some embodiments, the extent of conjugation ranges from 50-100%. In some embodiments, the extent of conjugation of from 70-100%. In some embodiments, the extent of conjugation of from 75-100%. In some embodiments, the extent of conjugation is between 77-78%. In certain embodiments, the extent of conjugation is 92-93%. In some embodiments, the extent of conjugation is 99-100%. In some embodiments, the extent of conjugation is 88-89%.

In some embodiments, the average effective diameters of the inventive polymers are between 100 and 10,000 nm. In some embodiments, the average effective diameters of the inventive polymers are between 100 and 5,000 nm. In some embodiments, the average effective diameters of the inventive polymers are between 200 and 2,000 nm. In some embodiments, the average effective diameters of the inventive polymers are between 100 and 500 nm. In some embodiments, the average effective diameters of the inventive polymers are between 300 and 600 nm.

In some embodiments, the average polydispersity of the inventive polymers is less than 0.4. In some embodiments, the average polydispersity of the inventive polymers is less than 0.3. In some embodiments, the average polydispersity of the inventive polymers is less than 0.2. In some embodiments, the average polydispersity of the inventive polymers is less than 0.1. In some embodiments, the average polydispersity of the inventive polymers is less than 0.05. In some embodiments, the average polydispersity of the inventive polymers is less than 0.01.

In some embodiments, the observed coefficient of friction of the inventive polymers are comparable to those of synovial fluid. In some embodiments, the observed coefficient of friction of the inventive polymers is no more than 50-times that observed with synovial fluid; in some embodiments, the observed coefficient of friction is no more than 45, 40, 35, 30, 25, 20, 15, 10, 9, 8, 7, 6, 5, 4, 3 or 2 times that observed with synovial fluid. In some embodiments, the observed coefficient of friction is between 0.5 and 20 times that of synovial fluid. In some embodiments, the coefficient of friction is between 0.5 and 10 times that of synovial fluid. In some embodiments, the coefficient of friction is between 0.5 and 5 times that of synovial fluid. In some embodiments, the coefficient of friction is between 0.5 and 2 times that of synovial fluid. In some embodiments, the coefficient of friction is between 1 and 2 times that of synovial fluid. In some embodiments, the coefficient of friction measurements is identical to that of synovial fluid.

In some embodiments, inventive polymers are retained within the cavity, tissue, or synovial space site of administeration for anywhere between 1 day and 24 months. In some embodiments, inventive polymers are retained within the cavity, tissue, or synovial space site of administeration for anywhere between 1 day and 18 months. In some embodiments, inventive polymers are retained within the cavity, tissue, or synovial space site of administeration for anywhere between 1 day and 12 months. In some embodiments, inventive polymers are retained within the cavity, tissue, or synovial space site of administeration for anywhere between 1 day and 9 months. In some embodiments, inventive polymers are retained within the cavity, tissue, or synovial space site of administeration for anywhere between 1 week and 9 months. In some embodiments, inventive polymers are retained within the cavity, tissue, or synovial space site of administeration for anywhere between 1 month and 9 months. The degree of retention as appreciated by those of skill in the art can be assessed by a variety of means, for example, using a polymer suitably labeled for detection by spectroscopy methods known to those of skill in the art.

In some embodiments, inventive polymers biodegrade at a rate corresponding to a half life of 24 hrs, 3 days, 1 week, 1 month, 6 months, 3 years.

In some embodiments, inventive polymers may be designed to maintain the spacing between two joints within a clinically beneficial range as established in the art. Spacing between two joints can be observed by X-ray using methods known to those of ordinary skill in the art.

In some embodiments, inventive polymers may be selected or designed to have charge in order to enhance retention. In some embodiments, inventive polymers may be designed to include a positive charge in order to enhance interaction with cartilage. In some embodiments, inventive polymers include a polyethylene glycol (PEG) unit to enhance biocompatibility. In some embodiments, inventive polymers include lysine moieties to influence shape.

### A. Preparation of Polymers

The present invention encompasses any of these polymers. In addition, the present invention provides polymers that can be formed using two or more of the polymers described above. The present invention includes dendritic polymers such as PAMAM, glycerol, glycerol-hydroacids, lysine, etc. The polymer may result from the formation of a direct or indirect linkage between the two or more polymers. Examples of direct linkages include covalent bonds and non-covalent bonds. Examples of covalent bonds include, but are not limited to, ester bond, ether bond, urea bond, amide bond, carbonate, thiocarbonate, thiourea, carbamate bond, urethane bond, Schiff base bond, peptide ligation (e.g., thiozolidine, N-thiazolidine), and carbon-carbon bond. Examples of non-covalent bonds include, but are not limited to, ionic bond, metal ligand bond, metal chelation bond (e.g., calcium or barium coordinated by a carboxylic acid), hydrogen bond, hydrophobic, fluorophobic, and van der Waals bond. Examples of indirect linkages include, but are not limited to, connecting molecules such as polyethylene glycol, polyacrylic glycol and natural polysaccharides, that can optionally be substituted, for example, with maleimide, activated ester, carboxylic acid, amine, thiol, cysteine, amino acid, acrylate, methacrylate, ester aldehyde, or aldehyde groups.

### II. Polymers as Delivery Agents

Inventive polymers, which can be in the form of viscous liquids or gels, can be used also as delivery agents. For example, an inventive polymer can be used to deliver one or more substances at the location where the polymer is injected (or applied) (e.g., joint, intervertebral disc, urinary system, skin).

Substances that can be delivered using the inventive polymers include any molecule, agent or compound that is suitable to be delivered to a patient at the location where the inventive polymer is to be injected or applied. For example, a suitable substance may be one or more of a growth factor, a cytokine, a small molecule, an analgesic, an anesthetic, an antimicrobial agent, an antibacterial agent, an antiviral agent, an antifungal agent, an antibiotic, an anti-inflammatory agent, an antioxidant, and an antiseptic agent.

Association between the polymer and substance may be covalent or non-covalent, direct or through a linker (e.g., a bifunctional agent). The association may be achieved by taking advantage of functional groups present on the polymer and substance. As can be readily appreciated by those skilled in the art, a polymer may be associated with any number of substances, which can be identical or different. In certain embodiments, the association between the polymer and substance is such that, in vivo, the substance is released from the polymer.

### III. Uses and Applications of Inventive Polymers

New polymers disclosed herein can find various applications in the biotechnology, pharmaceutical and medical fields. For example, polymers of the present invention can be used in viscosupplementation, e.g., in the treatment of osteoarthritic or sport-injured knee joints. The polymers can also be used to lubricate a hip or knee joint where an implanted metal or polymer is in contact including metal-metal, polymer-polymer, polymer-metal, ceramic-ceramic, polymer-ceramic, metal-ceramic, polymer-tissue, metal-tissue, or ceramic-tissue. They can also be used as viscoelastics, for example in opththalmic surgery, as tissue space filler for cosmetic procedures or treatment of urinary incontinence, and as anti-adhesives for wound care.

Accordingly, the present invention provides methods which generally include administration of an effective amount of an inventive polymer, or a pharmaceutical composition thereof, to an individual in need thereof.

### A. Indications

### Viscosupplements

Polymers of the present invention can be used as viscosupplements. As already mentioned above, viscosupplementation is a procedure involving injection of gel-like substances (generally hyaluronates, HAs) into a joint to supplement the viscous properties of synovial fluid. HA injections have been found to relieve pain in many osteoarthritis patients, with HAs of higher molecular weights (i.e., higher viscosity) showing better efficacy than those with lower molecular weights (i.e., lower viscosity). However, due to their short lifetime within the joint (about a couple of days), hyaluronate preparations currently available have only limited long-term benefit to the patient and require injection of large quantities of preparation and/or repeated injections.

### Viscoelastics

Polymers of the present invention may find applications as viscoelastics useful in surgery. Viscoelastic agents used in surgery may perform a number of different functions, including, without limitation, maintenance and support of soft tissue, tissue manipulation, lubrication, tissue protection, and adhesion prevention. As will be appreciated by one skilled in the art, the rheological properties of the polymers will necessarily affect their ability to perform these functions, and, as a result, their suitability for certain surgical procedures.

Viscoelastics are, for example, used in opththalmic surgery, such as cataract surgery. Cataracts, which are opacities of the natural ocular lens, can strike people in their 40s and 50s, but they occur most commonly in those over age 60—with a rapid increase in prevalence after that. More than 50% of all Americans 65 and older have cataracts, increasing to 70% among those over 75. In order to improve eyesight, the cataractous lens is surgically removed from the eye and an artificial intraocular lens is inserted in its place. Viscoelastics were introduced in the early 1980s in response to the observation that, during cataract surgery, the underside of the cornea was often damaged due to contact with instruments, devices, fluid bubbles, and intraocular lenses. Because the cells in this region cannot regrow, there was a need to protect them. Thus, during these surgical procedures, viscoelastic materials are typically injected into the anterior chamber of the eye to prevent collapse of the anterior chamber and to protect the delicate eye tissues from damage resulting from physical manipulation. Viscoelastics also gently inflate spaces inside the eye, making it easier to maneuver various tools inside the eye.

Other examples of ocular surgery procedures that employ viscoelastics include trabeculectomy (i.e., glaucoma filtration surgery), and vitrectomy (i.e., replacement of the vitrous, a normally clear, gel-like substance that fills the center of the eye), which may be performed to clear blood and debris from the eye, to remove scar tissue, or to alleviate traction on the retina.

### Tissue Space Fillers

Polymers of the present invention may find applications as tissue space fillers in any of a wide variety of soft tissue augmentation procedures, including, but not limited to, reconstruction or cosmetic enhancement, treatment for stress urinary incontinence, and treatment of vocal cord problems (e.g., paralysis, atrophy or paresis).

### Reconstruction or Cosmetic Enhancement Procedures.

Tissue space fillers are used to correct deformities or to reconstruct areas that are missing or defective due to surgical intervention, trauma, disease, aging, or congenital condition. Examples of reconstruction or cosmetic enhancement procedures include, but are not limited to, dermal tissue augmentation; filling of lines, folds, wrinkles, minor facial depressions, cleft lips and the like, especially in the face and neck; correction of minor deformities due to aging or disease, including in the hands and feet, fingers and toes; dermal filling of sleep lines and expression lines; replacement of dermal and subcutaneous tissue lost due to aging; lip augmentation; filling of crow's feet and the orbital groove around the eye; breast augmentation; chin augmentation; augmentation of the cheek and/or nose; filling of indentations in the soft tissue, dermal or subcutaneous, due to, e.g., overzealous liposuction or other trauma; filling of acne or traumatic scars and rhytids; filling of nasolabial lines, nasoglabellar lines and infraoral lines.

### Urinary Incontinence.

Urinary incontinence is an underserved market: there are approximately 40 million people in the U.S. that suffer from urinary incontinence, yet there are only about 250,000 procedures performed each year. Collagen bulking agents are generally used to treat urinary incontinence. They are injected into tissue surrounding the urethra to tighten the urethral sphincter and stop urine from leaking. However, these agents require several injections across multiple appointments. They also have a poor cure rate of approximately 27% to 36%. If the procedure is successful, the success is only temporary as the collagen reabsorbs into the surrounding tissue. A carbon-bead based product (Durasphere™, Advanced UroScience, Inc., Saint Paul, Minn.) entered the market in 1999 with the promise of permanence (due to less degradation of the material) but clinical data have not supported those claims and the product appears to have similar performance to collagen. Q-Med AB (Uppsala, Sweden) recently introduced Zuidex™, an HA gel which is reinforced by the addition of dextranomer, that promises immediate effects and ease of administration. New biomaterials, such as the inventive dendritic polymers, could impact the market if they require less material, fewer injections and had better longevity.

### Vocal Cord Augmentation.

In vocal cord disorders such as paralysis, atrophy and paresis, one or both vocal cords are weakened and lack the ability to close and thus vibrate properly, resulting in a soft, breathy or weak voice. The affected cord may also allow food and liquids into the trachea or lungs causing difficulty with swallowing and coughing. Vocal cord paralysis may be caused by chest and neck surgery, brain injury, neck injury, lung or thyroid cancer, certain neurologic conditions, or a viral infection. In older people, vocal cord atrophy is a common problem affecting voice production. Standard treatments of vocal cord disorders include voice therapy and surgery. In surgery, doctors attempt to add bulk to the injured vocal cord by injecting a substance (e.g., fat or collagen) into the cord. This moves the injured cord closer to the non-injured cord, allowing for better contact and improved speech and swallowing. Other substances are being studied for vocal cord augmentation including silicone paste, Teflon paste, calcium hydroxylapatite, and hyaluronic acid.

### Anti-Adhesives

Polymers of the present invention may be used as anti-adhesives. Anti-adhesives are devices that keep tissues from abnormally joining together following surgery. These abnormal unions, called adhesions, may form between an incision in the abdominal wall and the small bowel after abdominal surgery, leading to chronic pain or even bowel obstruction. Adhesions also occur following gynecological surgery, resulting in fibrous scarring that may involve the uterus, bladder, bowel or ovaries and fallopian tubes, and that can, in the worst case, lead to infertility. A wide variety of approaches, including use of steroids, non-steroidal anti-inflammatory drugs and minimally invasive surgical techniques, have been used in an attempt to prevent adhesions. However, biodegradable barriers appear to be the most promising tools available for keeping adjacent organs separate following surgery (P. B. Arnold et al., Fertil. Steril., 2000, 73: 157-161). Examples of such barriers include, but are not limited to, anti-adhesive membranes that may be laid on localized areas of the peritoneum, such as Interceed Absorbable Adhesion Barrier (Johnson & Johnson Patient Care Inc., New Brunswick, N.J.); Preclude Surgical Membrane (E.L. Gore Co., Flagstaff, Ariz.) and Seprafilm Surgical Membrane (Genzyme, Cambridge, Mass.); and viscous gels, such as Hyskon (Pharmacia, Piscataway, N.J.); Sepracoat (Genzyme) and Intergel (Lifecore Biomedical, Inc., Chaska, Minn.). Additional uses and applications of the inventive polymers will be immediately apparent to those skilled in the art.

### B. Dosages and Administration

In a method of treatment of the present invention, an inventive polymer, or a pharmaceutical composition thereof, will generally be administered in such amounts and for such a time as is necessary or sufficient to achieve at least one desired result. As will be appreciated by one skilled in the art, the desired result may vary depending on the condition to be treated (e.g., osteoarthritis, cataract, dermal or subcutaneous tissue loss, urinary incontinence, or vocal cord disorder) and the purpose of the polymer (e.g., viscosupplementation, tissue augmentation, adhesion prevention, or soft tissue maintenance, support or protection). Thus, for example, in certain embodiments, a polymer of the present invention may be administered to the knee joint of a patient suffering from osteoarthritis in such amounts and for such a time that it provides pain relief, prevents or reduces swelling, prevents or reduces loss of motion of the joint and/or or improves motion of the joint. In other embodiments, a polymer of the present invention may be administered to the eye of a patient undergoing cataract surgery in such amounts that it allows maintenance and support of soft tissue, tissue manipulation, lubrication, tissue protection, or adhesion prevention. In yet other embodiments, a polymer of the present invention may be administered to the skin of a patient undergoing a cosmetic procedure in such amounts and for such a time that lines, folds, wrinkles or minor facial depressions are filled.

A treatment according to the present invention may consist of a single dose or a plurality of doses over a period of time. Administration may be one or multiple times daily, weekly (or at some other multiple day interval) or on an intermittent schedule. The exact amount of an inventive polymer, or a pharmaceutical composition thereof, to be administered will vary from subject to subject and will depend on several factors (see below).

Polymers of the present invention, or pharmaceutical compositions thereof, may be administered using any route of administration effective for achieving the desired effect. Administration will generally be local rather than systemic. Methods of local administration include, but are not limited to, dermal, intradermal, intramuscular, intraperitoneal, subcutaneous, ocular, and intra-articular routes.

Depending on the route of administration, effective doses may be calculated according to the body weight, body surface area, or organ size of the subject to be treated. Optimization of the appropriate dosages can readily be made by one skilled in the art in light of pharmacokinetic data observed in human clinical trials. Alternatively or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

### C. Combination Therapies

It will be appreciated that methods of treatment of the present invention can be employed in combination with additional therapies (i.e., a treatment according to the present invention can be administered concurrently with, prior to, or subsequently to one or more desired therapeutics or medical procedures). The particular combination of therapies (therapeutics or procedures) to employ in such a combination regimen will take into account compatibility of the desired therapeutics and/or procedures and the desired therapeutic effect to be achieved.

Thus, for example, in methods where a polymer of the present invention is administered as a viscosupplement to a patient suffering from osteoarthritis, the patient may further receive a non-steroidal or steroidal anti-inflammatory drug and/or may undergo physical therapy. Alternatively or additionally, the inventive polymer may be administered in combination with another viscosupplement, e.g., hyaluronate, chitosan. Alternatively or additionally, the inventive polymer may be administered in combination with another aqueous soluble polymer, e.g., PEG, PEO, PAA. Thus, for example, in methods where a dendritic polymerpolymer of the present invention may be administered in combination with with another aqueous soluble polymer, e.g., PEG, PEO, PAA.

In many methods of the present invention, an inventive polymer is administered as part of a surgical or clinical procedure. For example, a polymer used as a viscoelastic agent may be administered during cataract surgery. An inventive polymer used as a tissue space filler may be administered during surgery for the treatment of urinary incontinence, during a tissue augmentation procedure for treatment of vocal cord problems, or during a cosmetic procedure, e.g., for wrinkle filling. An inventive polymer used as an anti-adhesive agent may be administered during abdominal or gynecologic surgery to prevent formation of adhesions following surgery.

### IV. Pharmaceutical Compositions Comprising Polymers

As mentioned above, methods of treatment of the present invention include administration of an inventive polymer per se or in the form of a pharmaceutical composition. A pharmaceutical composition will generally comprise an effective amount of at least one inventive polymer and at least one pharmaceutically acceptable carrier or excipient.

Pharmaceutical compositions of the present invention may be formulated according to general pharmaceutical practice (see, for example, “Remington's Pharmaceutical Sciences” and “Encyclopedia of Pharmaceutical Technology”, J. Swarbrick, and J. C. Boylan (Eds.), Marcel Dekker, Inc: New York, 1988). The optimal pharmaceutical formulation can be varied depending upon the route of administration and desired dosage. Such formulations may influence the physical state, stability, rate of in vivo release, and rate of in vivo clearance of the administered compounds. Formulation will preferably produce liquid or semi-liquid (e.g., gel) pharmaceutical compositions.

Pharmaceutical compositions may be formulated in dosage unit form for ease of administration and uniformity of dosage. The expression “unit dosage form”, as used herein, refers to a physically discrete unit of dendritic polymerpolymer for the patient to be treated. Each unit contains a predetermined quantity of active material calculated to produce the desired effect. It will be understood, however, that the total dosage of the composition will be decided by the attending physician within the scope of sound medical judgment.

Formulation of pharmaceutical compositions of the present invention will mainly depend on the form of administration chosen. In certain embodiments, injectable formulations (e.g., solutions, dispersions, suspensions, emulsions) will be preferred, for example, for administration to a joint (e.g., knee), an intervertebral disc, the urinary system, or the vocal cord. Injectable formulations can also be used for certain reconstruction or cosmetic procedures. Other procedures may alternatively use gels, lotions, creams, ointments, plasters, bandages, sheets, foams, films, sponges, dressings, or bioadsorbable patches that can be applied to the area in need of treatment.

### Formulation

Physiologically acceptable carriers, vehicles, and/or excipients for use with pharmaceutical compositions of the present invention can be routinely selected for a particular use by those skilled in the art. These include, but are not limited to, solvents, buffering agents, inert diluents or fillers, suspending agents, dispersing or wetting agents, preservatives, stabilizers, chelating agents, emulsifying agents, anti-foaming agents, ointment bases, penetration enhancers, humectants, emollients, and skin protecting agents.

Examples of solvents include water, Ringer's solution, U.S.P., isotonic sodium chloride solution, alcohols, vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, and diethylamine. Suitable suspending agents include, for example, naturally-occurring gums (e.g., acacia, arabic, xanthan, and tragacanth gum), celluloses (e.g., carboxymethyl-, hydroxyethyl-, hydroxypropyl-, and hydroxypropylmethylcellulose), alginates and chitosans. Examples of dispersing or wetting agents are naturally-occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate).

Preservatives may be added to a pharmaceutical composition of the present invention to prevent microbial contamination that can affect the stability of the formulation and cause infection in the patient. Suitable examples of preservatives include parabens (such as methyl-, ethyl-, propyl-, p-hydroxy-benzoate, butyl-, isobutyl- and isopropyl-paraben), potassium sorbate, sorbic acid, benzoic acid, methyl benzoate, phenoxyethanol, bronopol, bronidox, MDM hydantoin, iodopropylnyl butylcarbamate, benzalconium chloride, cetrimide, and benzylalcohol. Examples of chelating agents include sodium EDTA and citric acid.

Examples of emulsifying agents are naturally-occurring gums, naturally-occurring phosphatides (e.g., soybean lecithin, sorbitan mono-oleate derivatives), sorbitan esters, monoglycerides, fatty alcohols, and fatty acid esters (e.g., triglycerides of fatty acids). Anti-foaming agents usually facilitate manufacture, they dissipate foam by destabilizing the air-liquid interface and allow liquid to drain away from air pockets. Examples of anti-foaming agents include simethicone, dimethicone, ethanol, and ether.

Examples of gel bases or viscosity-increasing agents are liquid paraffin, polyethylene, fatty oils, colloidal silica or aluminum, glycerol, propylene glycol, carboxyvinyl polymers, magnesium-aluminum silicates, hydrophilic polymers (such as, for example, starch or cellulose derivatives), water-swellable hydrocolloids, carragenans, hyaluronates, and alginates. Ointment bases suitable for use in the pharmaceutical compositions of the present invention may be hydrophobic or hydrophilic; and specific examples include paraffin, lanolin, liquid polyalkylsiloxanes, cetanol, cetyl palmitate, vegetable oils, sorbitan esters of fatty acids, polyethylene glycols, and condensation products between sorbitan esters of fatty acids, ethylene oxide (e.g., polyoxyethylene sorbitan monooleate), and polysorbates.

Examples of humectants are ethanol, isopropanol glycerin, propylene glycol, sorbitol, lactic acid, and urea. Suitable emollients include cholesterol and glycerol. Examples of skin protectants include vitamin E, allatoin, glycerin, zinc oxide, vitamins, and sunscreen agents.

In certain embodiments, pharmaceutical compositions of the present invention may, alternatively or additionally, comprise other types of excipients including, thickening agents, bioadhesive polymers, and permeation enhancing agents. Thickening agents are generally used to increase viscosity and improve bioadhesive properties of pharmaceutical compositions. Examples of thickening agents include, but are not limited to, celluloses, polyethylene glycol, polyethylene oxide, naturally occurring gums, gelatin, karaya, pectin, alginic acid, and povidone. In certain embodiments, a thickening agent is selected for its thioxotropic properties (i.e., has a viscosity that is decreased by shaking or stirring). The presence of such as an agent in a pharmaceutical composition allows the viscosity of the composition to be reduced at the time of administration to facilitate its application, e.g., to a skin area to be repaired, and to increase after application so that the composition remains at the site of administration.

Permeation enhancing agents are vehicles containing specific agents that affect the delivery of active components through the skin. Permeation enhancing agents are generally divided into two classes: solvents and surface active compounds (amphiphilic molecules). Examples of solvent permeation enhancing agents include alcohols (e.g., ethyl alcohol, isopropyl alcohol), dimethyl formamide, dimethyl sulfoxide, 1-dodecylazocyloheptan-2-one, N-decyl-methylsulfoxide, lactic acid, N,N-diethyl-m-toluamide, N-methyl pyrrolidone, nonane, oleic acid, petrolatum, polyethylene glycol, propylene glycol, salicylic acid, urea, terpenes, and trichloroethanol. The surfactant permeation enhancing agent in the present inventive pharmaceutical compositions may be nonionic, amphoteric, cationic, anionic, or zwitterionic. Suitable nonioinic surfactants include poly(oxyethylene)-poly(oxypropylene) block copolymers, commercially known as poloxamers; ethoxylated hydrogenated castor oils; polysorbates, such as Tween 20 or Tween 80. Amphoteric surfactants include quaternized imidazole derivatives, cationic surfactants include cetypyridinium chloride, cationic surfactants include “soap” (fatty acid), alkylsulfonic acid salts (the main component of synthetic detergent, such as linear alkyl benzene sulfonate (LAS)), fatty alcohol sulfate (the main component of shampoo or old neutral detergents), and zwitterionic surfactants include the betaines and sulfobetaines. Injectable formulations can be sterilized, for example, by filtration through a bacterial-retaining filter, GAMA irradiation sterilization, E-Beam irradiation sterilization or by incorporating sterilizing agents in the form of sterile solid compositions which can be dissolved or dispersed in sterile water or other sterile injectable medium prior to use.

### Bioactive Agents

In certain embodiments, the inventive polymer(s) is(are) the only active ingredient(s) in an inventive pharmaceutical composition. In other embodiments, the pharmaceutical composition further comprises one or more bioactive agents. As already mentioned above, a bioactive agent may be associated with the inventive polymer. Alternatively or additionally, a bioactive agent may be added to the composition of polymer and does not form any associations with the polymer. As will be appreciated by one skilled in the art, selection of one or more bioactive agents as component(s) of an inventive pharmaceutical composition will be based on the intended purpose of the pharmaceutical composition (e.g., use in viscosupplementation in the treatment of joints, use as viscoelastics in cataract surgery, use as tissue space fillers for cosmetic procedures, treatment of urinary incontinence or treatment of vocal cord problems, or use as anti-adhesives for wound care). In general, the amount of bioactive agent present in an inventive pharmaceutical composition will be the ordinary dosage required to obtain the desired result through local administration. Such dosages are either known or readily determined by the skilled practitioner in the pharmaceutical and/or medical arts. Examples of bioactive agents that can be present in a pharmaceutical composition of the present invention include, but are not limited to, analgesics, anesthetics, pain-relieving agents, antimicrobial agents, antibacterial agents, antiviral agents, antifungal agents, antibiotics, anti-inflammatory agents, antioxidants, antiseptic agents, antipruritic agents, immunostimulating agents, and dermatological agents. Specific examples of suitable bioactive agents are provided and discussed below.

### Pain Relieving Agents.

A bioactive agent may be selected for its ability to prevent or alleviate pain, soreness or discomfort, to provide local numbness or anesthesia, and/or to prevent or reduce acute post-operative surgical pain. Thus, suitable pain relieving agents include, but are no limited to, compounds, molecules or drugs which, when applied locally, have a temporary analgesic, anesthetic, numbing, paralyzing, relaxing or calming effect.

Analgesics suitable for use in the present invention include non-steroidal, anti-inflammatory drugs (NSAIDs). NSAIDs have analgesic, antipyretic and anti-inflammatory activity. They act peripherally to provide their analgesic effect by interfering with the synthesis of prostaglandin, through cyclooxygenase (COX) inhibition. There are many different types of NSAIDs, including aspirin and other salicylates. Examples include, but are not limited to, ibuprofen, naproxen, sulindac, diclofenac, piroxicam, ketoprofen, diflunisal, nabumetone, etodolac, oxaprozin, and indomethacin. Aspirin is anti-inflammatory when administered in high doses, otherwise it is just a pain killer like acetaminophen. Acetaminophen has similar analgesic and antipyretic effects to the NSAIDs, but does not provide an anti-inflammatory effect. Several of the more potent NSAIDs have been developed into topical products for local administration to painful areas of the body.

Analgesics suitable for use in the present invention also include opioids. As used herein, the term “opioid” refers to any agonists or antagonists of opioid receptors such as the μ-, κ-, and δ-opioid receptors and different subtypes. Examples of opioids include, but are not limited to, alfentanil, allylprodine, alphaprodine, amiphenazole, anileridine, benzeneacetamine, benzoylhydrazone, benzylmorphine, benzitramide, nor-binaltorphimine, bremazocine, buprenorphine, butorphanol, clonitazene, codeine, cyclazocine, desomorphine, dextromoramide, dezocine, diampromide, dihydrocodeine, dihydrocodeine enol acetate, dihydromorphine, dimenoxadol, dimepheptanol, dimethylthiambutene, dioxaphetyl butyrate, dipipanone, diprenorphine, eptazocine, ethoheptazine, ethylketocyclazocine, ethylmethylthiambutene, etonitazene, etorphine, fentanyl, hydrocodone, hydromorphone, hydroxypethidine, isomethadone, ketobemidone, levallorphan, levorphanol, lofentanil, loperamide, meperidine, meptazinol, metazocaine, methadone, metopon, morphine, morphiceptin, myrophine, nalbuphine, nalmefene, nalorphine, naltrindole, naloxone, naltrexone, narceine, nicomorphine, norlevorphanol, normethadone, normorphine, norpipanone, opium, oxycodone, oxymorphone, papaveretum, papaverine, pentazocine, phenadoxone, phenazocine, phenoperidine, piminodine, piperidine, pirtramide, proheptazine, promedol, propiram, propoxyphene, remifentanil, spiradoline, sufentanil, tilidine, trifluadom, and active derivatives, prodrugs, analogs, pharmaceutically acceptable salts, or mixtures thereof. Examples of peptide opioids include, but are not limited to, [Leu⁵]enkephalin, [Met⁵]enkephalin, DynorphinA, Dynorphin B, α-Neoendorphin, β-Neoendorphin, βₕ-Endorphin, Deltorphin II, Morphiceptin, and active derivatives, analogs, pharmaceutically acceptable salts, or mixtures thereof.

Tricyclic antidepressants can be useful as adjuvant analgesics. They are known to potentiate the analgesic effects of opioids (V. Ventafridda et al., Pain, 1990, 43: 155-162) and to have innate analgesic properties (M. B. Max et al., Neurology, 1987, 37: 589-596; B. M. Max et al., Neurology, 1988, 38: 1427-1432; R. Kishore-Kumar et al., Clin. Pharmacol. Ther., 1990, 47: 305-312). Tricyclic antidepressants include, but are not limited to, amitriptyline, amoxapine, clomipramine, desipramine, doxepin, imipramine, nortriptyline, protriptyline, and trimipramine.

Anesthetics that are suitable for use in the practice of the present invention include sodium-channel blockers. Examples of sodium-channel blockers include, but are not limited to, ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, etidocaine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, and active derivatives, prodrugs, analogs, pharmaceutically acceptable salts, or mixtures thereof.

Local anesthetics with different pharmacodynamics and pharmacokinetics may be combined in an inventive pharmaceutical composition in order to improve the effectiveness and tolerance of the composition. For example, an inventive composition may comprise an euctectic mixture of lidocaine and prilocaine, or a mixture of lidocaine and tetracaine. It has been reported (see, for example, U.S. Pat. Nos. 5,922,340 and 6,046,187) that co-administration of a glucocorticosteroid and a local anesthetic may prolong or otherwise enhance the effect of local anesthetics. Examples of glucocorticosteroids include dexamethazone, cortisone, hydrocortisone, prednisone, prednisolone, beclomethasone, betamethasone, flunisolide, fluocinolone, acetonide, fluocinonide, triamcinolone, and the like.

Locally acting vasoconstructive agents are also known to provide effective enhancement of local anesthesia, especially when administered through controlled release. Examples of vasoconstrictor agents include, but are not limited to, catechol amines (e.g., epinephrine, norepinephrine and dopamine); metaraminol, phenylephrine, sumatriptan and analogs, alpha-1 and alpha-2 adrenergic agonists, such as, for example, clonidine, guanfacine, guanabenz, and dopa (i.e., dihydroxyphenylalanine), methyldopa, ephedrine, amphetamine, methamphetamine, methylphenidate, ethylnorepinephrine ritalin, pemoline, and other sympathomimetic agents.

### Anti-Infective Agents.

Anti-infective agents for use in pharmaceutical compositions of the present invention are compounds, molecules or drugs which, when administered locally, have an anti-infective activity (i.e., they can decrease the risk of infection; prevent infection; or inhibit, suppress, combat or otherwise treat infection). Anti-infective agents include, but are not limited to, antiseptics, antimicrobial agents, antibiotics, antibacterial agents, antiviral agents, antifungal agents, anti-protozoan agents, and immunostimulating gents.

Antiviral agents suitable for use in the present invention include RNA synthesis inhibitors, protein synthesis inhibitors, immunostimulating agents, and protease inhibitors. Antiviral agents may, for example, be selected from the group consisting of acyclovir, amantadine hydrochloride, foscarnet sodium, ganeiclovir sodium, phenol, ribavirin, vidarabine, and zidovudine.

Examples of suitable antifungal agents include lactic acid, sorbic acid, Amphotericin B, Ciclopirox, Clotrimazole, Enilconazole, Econazole, Fluconazole, Griseofulvin, Halogropin, Introconazole, Ketoconazole, Miconazole, Naftifine, Nystatin, Oxiconazole. Sulconazole, Thiabendazole, Terbinafine, Tolnaftate, Undecylenic acid, Mafenide, Silver Sulfadiazine, and Carbol-Fushsin.

Antibiotics and other antimicrobial agents may be selected from the group consisting of bacitracin; the cephalosporins (such as cefadroxil, cefazolin, cephalexin, cephalothin, cephapirin, cephradine, cefaclor, cefamandole, cefonicid, ceforanide, cefoxitin, cefuroxime, cefoperazone, cefotaxime, cefotetan, ceftazidime, ceftizoxime, ceftriaxone, and meropenem); cycloserine; fosfomycin, the penicillins (such as amdinocillin, ampicillin, amoxicillin, azlocillin, bacamipicillin, benzathine penicillin G, carbenicillin, cloxacillin, cyclacillin, dicloxacillin, methicillin, mezlocillin, nafcillin, oxacillin, penicillin G, penicillin V, piperacillin, and ticarcillin); ristocetin; vancomycin; colistin; novobiocin; the polymyxins (such as colistin, colistimathate, and polymyxin B); the aminoglycosides (such as amikacin, gentamicin, kanamycin, neomycin, netilmicin, paromomycin, spectinomycin, streptomycin, and tobramycin), the tetracyclines (such as demeclocycline, doxycycline, methacycline, minocycline, and oxytetracycline); carbapenems (such as imipenem); monobactams (such as aztreonam); chloramphenicol; clindamycin; cycloheximide; fucidin; lincomycin; puromycin; rifampicin; other streptomycins; the macrolides (such as erythromycin and oleandomycin); the fluoroquinolones; actinomycin; ethambutol; 5-fluorocytosine; griseofulvin; rifamycins; the sulfonamides (such as sulfacytine, sulfadiazine, sulfisoxazole, sulfamethoxazole, sulfamethizole, and sulfapyridine); and trimethoprim.

Other antibacterial agents include, but are not limited to, bismuth containing compounds (such as bismuth aluminate, bismuth subcitrate, bismuth subgalate, and bismuth subsalicylate); nitrofurans (such as nitrofurazone, nitrofurantoin, and furozolidone); metronidazole; tinidazole; nimorazole; and benzoic acid.

Antiseptic agents may be selected from the group consisting of benzalkonium chloride, chlorhexidine, benzoyl peroxide, hydrogen peroxide, hexachlorophene, phenol, resorcinol, and cetylpyridinium chloride.

The risk of infection is directly influenced by a suppressed immune system due to disease or medication. Immunostimulating agents are compounds, molecules or drugs that stimulate the immune system of a patient to respond to the presence of a foreign body, for example, by sending macrophages to the infected site(s). Immunostimulating agents suitable for use in the present invention may be selected from a wide range of therapeutic agents, such as interleukin 1 agonists, interleukin 2 agonists, interferon agonists, RNA synthesis inhibitors, and T cell stimulating agents.

### Anti-Inflammatory Agents.

Anti-inflammatory agents for use in pharmaceutical compositions of the present invention are compounds, molecules or drugs which, when administered locally, have an anti-inflammatory activity (i.e., they can prevent or reduce the duration and/or severity of inflammation; prevent or reduce injury to cells at the injured/damaged site; prevent or reduce damage or deterioration of surrounding tissue due to inflammation; and/or provide relief from at least one of the manifestations of inflammation such as erythema, swelling, tissue ischemia, itching, fever, scarring, and the like).

Anti-inflammatory agents include NSAIDs and steroidal anti-inflammatory agents. Examples of NSAIDs can be found above. Examples of steroidal anti-inflammatory agents include, but are not limited to, aclomethasone dipropionate, flunisolide, fluticasone, budesonide, triamcinolone, triamcinoline acetonide, beclomethasone diproprionate, betamethasone valerate, betamethasone diproprionate, hydrocortisone, cortisone, dexamethason, mometasone furoate, prednisone, methylprednisolone aceponate, and prednisolone.

Anti-inflammatory agents may, alternatively or additionally, be selected from the wide variety of compounds, molecules, and drugs exhibiting antioxidant activity. Antioxidants are agents that can prevent or reduce oxidative damage to tissue. Examples of antioxidants may include, but are not limited to, vitamin A (retinal), vitamin B (3,4-didehydroretinol), vitamin C (D-ascorbic acid, L-ascorbic acid), α-carotene, β-carotene, γ-carotene, δ-carotene, vitamin E (α-tocopherol), β-tocopherol, γ-tocopherol, δ-tocopherol, tocoquinone, tocotrienol, butylated hydroxy anisole, cysteine, and active derivatives, analogs, precursors, prodrugs, pharmaceutically acceptable salts or mixtures thereof.

### Other Bioactive Agents

In certain embodiments, the bioactive agent is a biomolecule that is naturally present in the body and/or that is naturally secreted at an injured or damaged site (i.e., body area) and plays a role in the natural healing process. As will be apparent to those of ordinary skill in the art, variants, synthetic analogs, derivatives, and active portions of these biomolecules can, alternatively, be used in the inventive compositions as long as they exhibit substantially the same type of property/activity as the native biomolecule. Such variants, synthetic analogs, derivatives or active portions are intended to be within the scope of the term “bioactive agents”. Bioactive biomolecules may be extracted from mammalian tissues and used in inventive pharmaceutical compositions either crude or after purification. Alternatively, they may be prepared chemically or by conventional genetic engineering techniques, such as via expression of synthetic genes or of genes altered by site-specific mutagenesis.

Examples of suitable bioactive biomolecules include cytokines and growth factors. Cytokines and growth factors are polypeptide molecules that regulate migration, proliferation, differentiation and metabolism of mammalian cells. A diverse range of these biomolecules have been identified as potentially playing an important role in regulating healing. Examples of cytokines include, but are not limited to, interleukins (ILs) (e.g., IL-1, IL-2, IL-4 and IL-8), interferons (IFNs) (e.g., IFN-α, IFN-β, and IFN-γ), and tumor necrosis factors (e.g., TNF-α), or any variants, synthetic analogs, active portions or combinations thereof. Examples of growth factors include, but are not limited to, epidermal growth factors (EGFs), platelet-derived growth factors (PDGFs), heparin binding growth factor (HBGFs), fibroblast growth factors (FGFs), vascular endothelial growth factors (VEGFs), insulin-like growth factors (IGFs), connective tissue activating peptides (CTAPs), transforming growth factors alpha (TGF-α) and beta (TGF-β), nerve growth factor (NGFs), colony stimulating factors (G-CSF and GM-CSF), and the like, or any variants, synthetic analogs, active portions or combinations thereof.

Other examples of suitable bioactive biomolecules include proteoglycans, or portions thereof. Proteoglycans are protein-carbohydrate complexes characterized by their glycosaminoglycan (GAG) component. GAGs are highly charged sulfated and carboxylated polyanionic polysaccharides. Examples of GAGs suitable for use in pharmaceutical compositions of the present invention include, but are not limited to, hyaluronan, chondroitin sulfate, dermatan sulfate, heparan sulfate, and keratan sulfate.

Still other examples of suitable bioactive biomolecules include adhesion molecules. Adhesion molecules constitute a diverse family of extracellular and cell surface glycoproteins involved in cell-cell and cell-extracellular matrix adhesion, recognition, activation, and migration. Adhesion molecules are essential to the structural integrity and homeostatic functioning of most tissues, and are involved in a wide range of biological processes, including embryogenesis, inflammation, thrombogenesis, and tissue repair. Adhesion molecules include matricellular proteins (e.g., thrombospondins and tenascins), and cell surface adhesion molecules (e.g., integrins, selectins, cadherins, and immunoglobulins).

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

All reactions were carried out at room temperature in oven-dried glassware. All solvents were distilled prior to use. Gel permeation chromatography (GPC) was performed either with tetrahydrofuran (THF) as eluent through a Waters HR-5/HR-5E organic column series or with water as eluent through a Shodex-OH column. All molecular weights were measured against polystyrene standards for THF soluble polymers and against dextran standards for water soluble polymers. Proton NMR spectra were recorded on a Varian Inova 4000 MHz spectrometer, chemical shifts are reported downfield from tetramethylsilane in parts per million. Broad or overlapping peaks, often observed in the spectra of polymers are denoted “br” below.

### Example 1

Synthesis of 2K PEG Acid. 2,000 molecular weight methoxy poly(ethylene glycol) (5.00 g, 0.0025 mol), succinic anhydride (1.25 g, 0.0125 mol), and 4-dimethylaminopyridine (0.031 g, 0.00025 mol) were reacted in 12.5 mL of pyridine in a 100 mL round bottom flask. The reaction was left overnight and then precipitated in 75 mL of diethylether.

### Example 2

Synthesis of 5K PEG Acid. 5,000 molecular weight methoxy poly(ethylene glycol) (10.00 g, 0.002 mol), succinic anhydride (1.00 g, 0.010 mol), and 4-dimethylaminopyridine (0.031 g, 0.0020 mol) were reacted in 10.0 mL of pyridine in a 100 mL round bottom flask. The reaction was left overnight and then precipitated in 125 mL of diethylether.

### Example 3

Synthesis of N-Hydroxysuccinimide (NHS) PEG Ester. 2,000 molecular weight methoxy poly(ethylene glycol) acid (0.500 g, 0.000238 mol), dicyclohexylcarbodiimide (0.0540 g, 0.000262 mol), and N-hydroxysuccinimide (0.0411 g, 0.000357 mol) were reacted in a 50 mL round bottom flask. The reaction was run for 24 hours. After purification, the N-hydroxysuccinimide poly(ethylene glycol) ester (0.075 g, 0.0000341 mol) and 6.73 μL of polyamidoamine generation 2 dendrimer (0.00579 g, 0.00000178 mol) were reacted in 1 mL of borate buffer (pH=9.8) for 24 hours.

### Example 4

Synthesis of Pentafluorophenol (PFP) Ester. 2,000 molecular weight methoxy poly(ethylene glycol) acid (0.200 g, 0.0000952 mol), dicyclohexylcarbodiimide (0.0393 g, 0.000190 mol), 6 and pentafluorophenol (0.0529 g, 0.000286 mol) were reacted in a 50 mL round bottom flask. The reaction was run for 24 hours. After purification, the pentafluorophenol poly(ethylene glycol) ester (0.0065 g, 0.00000296 mol) and 0.583 μL of polyamidoamine generation 2 dendrimer (0.0000508 g, 0.000000154 mol) were reacted in 1 mL of borate buffer (pH=9.8) for 24 hours.

### Example 5

Synthesis of 20K Methoxy-Poly(Ethylene Glycol)-Nitrophenyl Carbonate. 20,000 molecular weight methoxy poly(ethylene glycol) (0.500 g, 0.0000247 mol) and 0.025 mL of polyamidoamine generation 3 (0.00051 g, 0.000000736 mol) were reacted in 1.5 mL of methylene chloride and left to run for 24 hours before purification with a 15 mL 30,000 molecular weight cutoff spin concentrator tube.

### Example 6

Coupling of 2K PEG-PAMAM G2. 2,000 methoxy poly(ethylene glycol) acid (2.064 g, 0.000983 mol), 1-ethyl-3-(3-dimethylaminopropyl)carbodiimide hydrochloride (0.377 g, 0.00197 mol), hydroxybenzotriazole (0.0235 g, 0.000154 mol), and 0.500 mL of polyamidoamine generation 2 (0.100 g, 0.0000307 mol) were reacted in 5.0 mL of methanol. The reaction was left to run for 24 hours before purification with a 15 mL 30,000 molecular weight cutoff spin concentrator tube.

### Example 7

Coupling of 5K PEG-PAMAM G2. 5,000 methoxy poly(ethylene glycol) acid (5.010 g, 0.000983 mol), 1-ethyl-3-(3-dimethylaminopropyl)carbodiimide hydrochloride (0.471 g, 0.00245 mol), hydroxybenzotriazole (0.0235 g, 0.000154 mol), and 0.500 mL of polyamidoamine generation 2 (0.100 g, 0.0000307 mol) were reacted in 5.0 mL of methanol. The reaction was left to run for 24 hours before purification with a 15 mL 30,000 molecular weight cutoff spin concentrator tube.

### Example 8

Coupling of 2K PEG-PAMAM G3. 2,000 methoxy poly(ethylene glycol) acid (1.945 g, 0.000926 mol), 1-ethyl-3-(3-dimethylaminopropyl)carbodiimide hydrochloride (0.355 g, 0.00185 mol), hydroxybenzotriazole (0.0222 g, 0.000145 mol), and 0.500 mL of polyamidoamine generation 3 (0.100 g, 0.0000145 mol) were reacted in 5.0 mL of methanol. The reaction was left to run for 24 hours before purification with a 15 mL 30,000 molecular weight cutoff spin concentrator tube.

### Example 9

Coupling of 5K PEG-PAMAM G3. 5,000 methoxy poly(ethylene glycol) acid (9.449 g, 0.00185 mol), 1-ethyl-3-(3-dimethylaminopropyl)carbodiimide hydrochloride (0.888 g, 0.00463 mol), hydroxybenzotriazole (0.0222 g, 0.000145 mol), and 1.00 mL of polyamidoamine generation 3 (0.200 g, 0.0000290 mol) were reacted in 5.0 mL of methanol. The reaction was left to run for 24 hours before purification with a 15 mL 30,000 molecular weight cutoff spin concentrator tube.

### Example 10

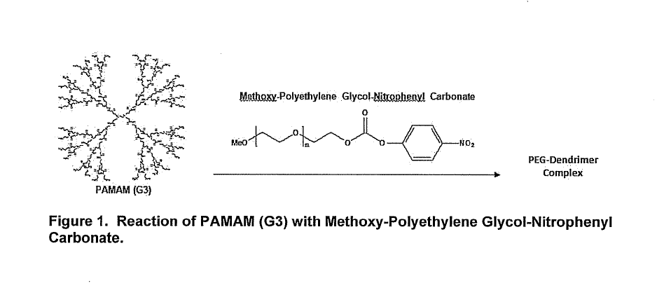

20,000 Methoxy-Poly(Ethylene Glycol)-Nitrophenyl Carbonate Reaction. A larger PEGylated dendrimer was created by coupling 20,000 mPEG to PAMAM G3. Because 20,000 molecular weight mPEG was not available in the lab, a 20,000 molecular weight PEG acid molecule could not be formed following the developed procedures outlined above. Consequently, an mPEG-nitrophenyl carbonate compound (FIG. 1 below) with a molecular weight of 20,000 was ordered from Laysan Bio, Inc. The nitrophenyl carbonate group is an excellent leaving group and thus, only needs to come in contact with the dendrimer to initiate coupling. Both reactants were added together along with one crystal of 4-dimethylaminopyridine (DMAP) in a dichloromethane (DCM) solvent and left to react overnight. After immediately reacting, the solution turned bright yellow, indicating the conversion of the nitrophenyl carbonate group on the mPEG to an ion in solution (FIG. 1).

### Example 11

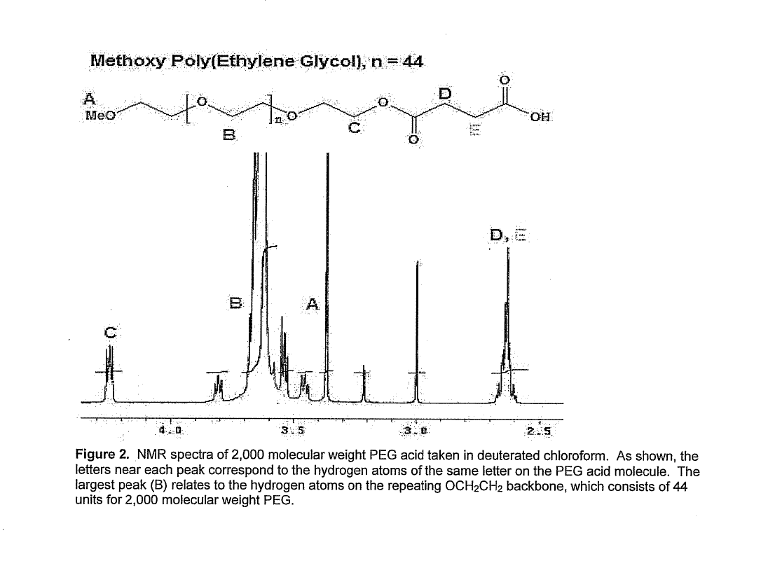

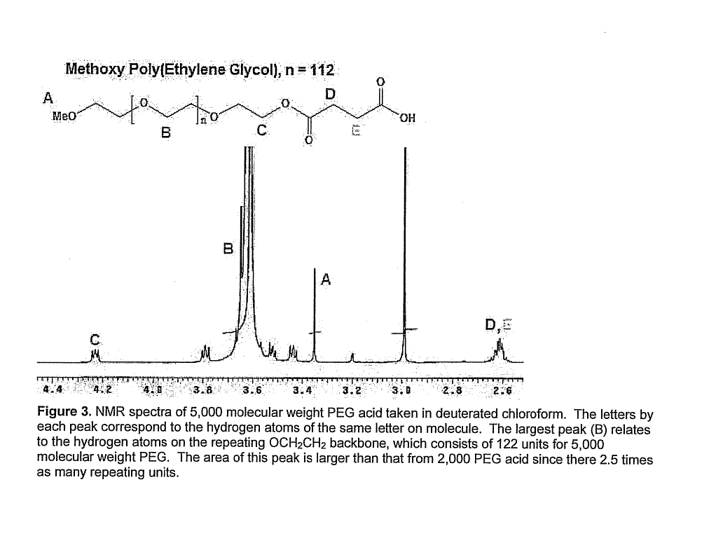

Nuclear Magnetic Resonance (NMR) Spectroscopy of Synthesized Lubricants. Nuclear magnetic resonance (NMR) spectroscopy was used both as a method for confirming the formation of the PEGylated PAMAM dendrimers after their synthesis and purification, and also as a technique for determining the molecular weights and percent conjugation of the terminal amine groups on PAMAM. Before any coupling reactions were implemented, reactions were run to form the intermediate PEG acid molecules (2,000 and 5,000 molecular weights) following the protocol outlined in the methods. After purification through precipitation in ether, an NMR of both molecular weight species was taken (FIGS. 2 and 3) to ensure that the acid group had formed. Each of the five types of hydrogen atoms were present in both spectra, including the hydrogen atoms contributed by succinic anhydride (Peak D,E), indicating that that the PEG acid could be used in the synthesis reactions.

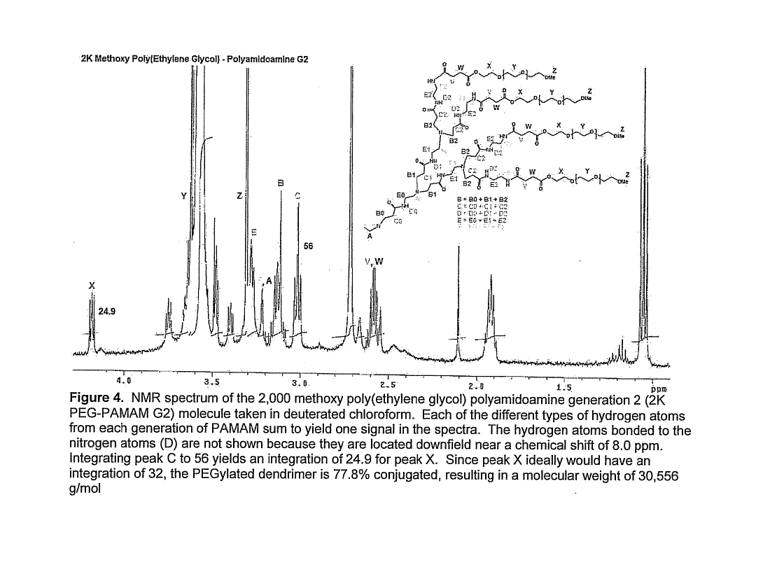

The proposed protocols to couple 2,000 and 5,000 molecular PEG acid to second and third generation PAMAM dendrimers and purify the resultant compounds were followed. NMR spectra were taken of these resultant dendritic compounds, with the first of four presented in FIG. 4. FIG. 4 depicts the spectrum for the product of the coupling reaction between 2,000 MW PEG acid and PAMAM G2, with the conjugated dendrimer product of this reaction abbreviated as 2K PEG-PAMAM G2. The spectrum contains peaks for hydrogen atoms from both the PEG acid and the PAMAM, indicating that conjugation had occurred. The peaks from the dendrimer have chemical shifts located between 2.9 and 3.4 ppm. The product is not assumed to be simply a mixture between unconjugated PAMAM and uncoupled PEG acid because the purification technique removed molecules possessing molecular weights below 30,000 g/mol.

The molecular weight of the 2K PEG-PAMAM G2 compound was predicted using peaks C (3.0 ppm) and X (4.3 ppm). Peak C represents the total number of hydrogen atoms (56) located near the carboxyl group in all generations of the PAMAM whereas peak X relates to the hydrogen atoms near the repeating backbone unit of the PEG. The dendrimer peak was integrated to 56, which made the corresponding relative integration of peak X calculated to be 24.9. If the dendrimer were to be completed conjugated, peak X would have an integration of 32. Consequently, the dendrimer contains 24.9/32 of these hydrogen atoms, or is 77.8% conjugated, with 13 of the 16 terminal amine groups being coupled to the 2,000 PEG. This results in a calculated molecular weight of 30,556 g/mol.

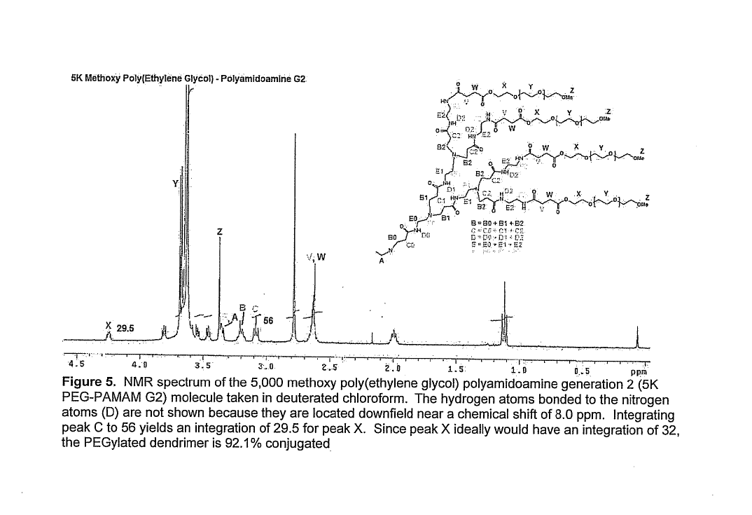

FIG. 5 depicts the NMR spectrum of the 5K PEG-PAMAM G2 product, which was formed from an EDC coupling reaction between 5,000 MW PEG acid and PAMAM G2. Following the same reasoning implemented in determining the molecular weight of the 2K PEG-PAMAM G2 product, the calculated molecular weight of the 5K PEG-PAMAM G2 compound is 79,756 g/mol. This corresponds to a percent conjugation of 92.1% and means that 15 of the 16 terminal amine groups of PAMAM G2 are coupled to PEG.

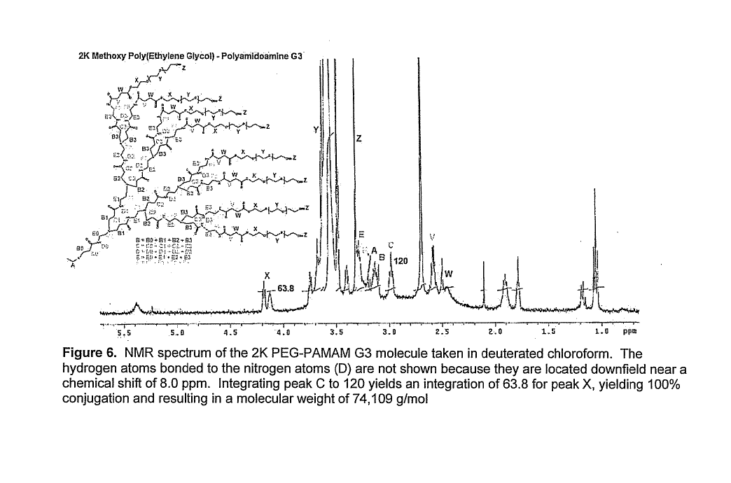

The corresponding NMR spectrum taken of the purified 2K PEG-PAMAM G3 product in deuterated chloroform appears in FIG. 6. The only difference between the distribution of hydrogen atoms of PAMAM G2 and PAMAM G3 in an NMR spectrum is that there are a greater number of each type of hydrogen atom. Correspondingly, the number of hydrogen atoms neighboring the carboxyl group is 120, instead of 56. Following this integration for peak C, the corresponding relative integration of peak X is 63.8. The integration would have been 64 had the dendrimer been completely conjugated; therefore, the resultant compound is 100% conjugated with all 16 terminal amine groups coupled to a 5,000 PEG molecular. The calculated molecular weight for 100% conjugation is 74,109 g/mol.

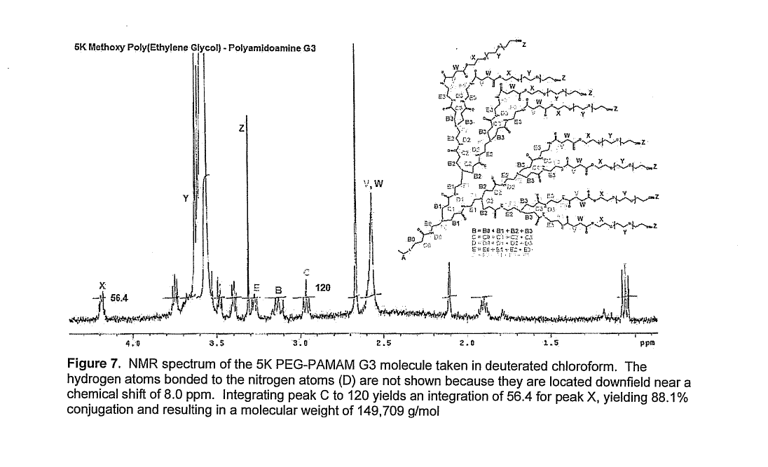

FIG. 7 shows the NMR spectrum for the 5K PEG-PAMAM G3 product, the fourth and final spectrum taken for the synthesized lubricants. The same calculation method used in determining the molecular weight of the 2K PEG-PAMAM G3 compound was also implemented: peak C (chemical shift=3.0 ppm) is integrated to an area of 120 and the corresponding relative integration of peak X (chemical shift=4.2 ppm) is 56.4. The percent conjugation is 56.4/64, or 88.1% conjugated. Such percentage relates to 28 of the 32 arms of the PAMAM G3 dendrimer being coupled to a PEG molecule. Therefore, the molecular weight of the 5K PEG-PAMAM G3 compound is calculated to be 149,709 g/mol.

The tabulated results for determination of the molecular weights of the synthesized compounds appear in Table 1. The data indicates that all four combinations of PEG and PAMAM dendrimer were successfully formed using the developed coupling protocol and that all exhibited greater than 75% conjugation of the terminal amine groups.

TABLE 1
Molecular weight predictions from NMR. All four of the synthesized
lubricants were formed by conjugation of more than 75% of the terminal
amine groups. The smallest compound, 2K PEG-PAMAM G2, exhibited
the lowest conjugation at 78% whereas the 5K PEG-PAMAM G3
exhibited that of essentially 100%.

| | | | Theoretical |
|---|---|---|---|
| | Predicted Molecular | Percent | Molecular |
| | Weight From NMR | Conjugation | Weight |
| Sample Name | (g/mol) | (%) | (g/mol) |
| 2K PEG-PAMAM G2 | 30,556 | 77.8 | 36,856 |
| 5K PEG-PAMAM G2 | 79,756 | 92.1 | 84,856 |
| 2K PEG-PAMAM G3 | 74,109 | 99.6 | 74,109 |
| 5K PEG-PAMAM G3 | 149,709 | 88.1 | 170,109 |

### Example 12

Dynamic Light Scattering (DLS) of Synthesized Lubricants. Diameter measurements of the synthesized lubricants obtained from dynamic light scattering (DLS) are listed in Table 2. Readings were also made for the unconjugated G2 and G3 dendrimers as standards to compare to the samples. Data was not collected for 2K and 5K PEG acids because their small size is beyond the detection limit of the particle analyzer. The results indicate that the compounds form some type of aggregates (micelles, clusters, etc.) when in solution due to the large differences in effective diameter. For example, the unconjugated G3 appears to aggregate since its diameter is nearly three times as wide as G2. Both the compounds containing 5K PEG have larger diameters than the other synthesized molecules, indicating that they randomly form more aggregates at concentrations of 3.33 mg/mL (all molecules were tested at this concentration). On the other hand, the 2K PEG-PAMAM G2 compound is smaller in size with a diameter of only 183.9 nm. This value reveals that the compounds can also collapse and do not have to remain in a fully extended, branching network. The molecules could also possibly collapse after conjugation, which would explain the lower diameters for the 2K PEG-PAMAM G2 and 2K PEG-PAMAM G3 compounds. Even the largest PEG molecule (20,000) only formed a 293.5 nm diameter compound, which presents further evidence for aggregation among the 5K PEG-containing compounds.

TABLE 2
Data collected from the dynamic light scattering measurements. All of the
examined compounds exhibited a fairly low polydispersity, with none
greater than 0.350. Ideally, the polydispersity should be close to zero.
Dynamic Light Scattering Data

| | Average Effective Diameter | |
|---|---|---|
| Sample Name | (nm) | Polydispersity |
| PAMAM G2 | 270.7 | 0.282 |
| PAMAM G3 | 661.0 | 0.250 |
| 2K PEG-PAMAM G2 | 183.9 | 0.005 |
| 5K PEG-PAMAM G2 | 508.9 | 0.345 |
| 2K PEG-PAMAM G3 | 212.3 | 0.254 |
| 5K PEG-PAMAM G3 | 838.9 | 0.347 |
| 20K PEG-PAMAM G3 | 293.5 | 0.190 |

### Example 13

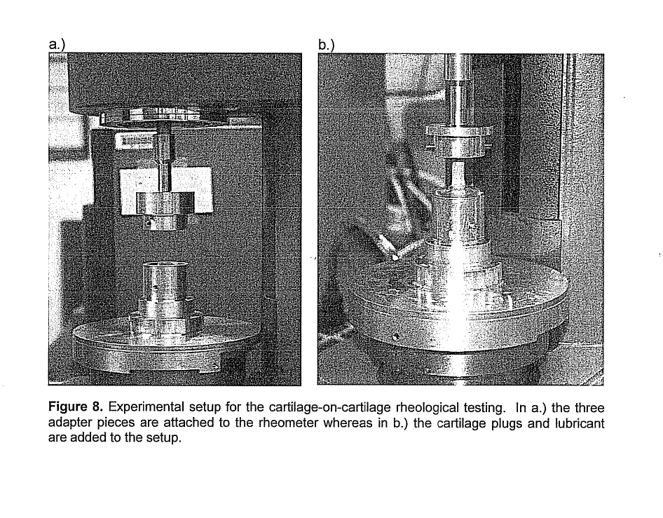

Rheology of Synthesized Lubricants. Rheological testing was used to determine the coefficient of friction for the synthesized lubricants in aluminum-on-steel contact. Measurements were made on an AR 1000 Controlled Strain Rheometer from TA Instruments equipped with a Peltier temperature control (FIGS. 8). 10 and 20% solutions of the samples were prepared by dissolving 100 and 200 mg of the compound in 1 mL of sterile Dulbecco's Phosphate Buffered Saline (DPBS) from Mediatech, Inc. For each test, the 1 mL solution was placed between the 40 mm diameter steel plate of the rheometer and a 40 mm diameter, 0° angle aluminum parallel plate adapter piece. A normal force of 5 N with a tolerance of 0.5 N was applied at a temperature of 25° C. for a torsion test in frequency sweep mode of the rheometer. In the rheometer computer software, the angular frequency range was set from 0.01 to 10 Hz, and the strain was given a constant, controlled value of 1%, or 0.01. The given frequencies generated an oscillatory torque (M) and oscillatory stress (σ_{S}), which are related by equation 1. The variable R is the radius of the steel plate (R=20 mm).

$${σ}_{s} = \frac{2 M}{π {R}^{3}} \qquad ( Equation 1 )$$

Measurements for the oscillatory and normal (σ_{N}) stresses of the compound for each angular frequency within the set range were determined and displayed by the AR Instrument Control computer software. Coefficient of friction (μ) data was calculated manually from the oscillatory stress and the normal stress, according to equation 2.

$$μ = \frac{{σ}_{s}}{{σ}_{N}} \qquad ( Equation 2 )$$

Coefficient of friction measurements for aluminum-on-steel contact were made by performing a torsion test in frequency sweep mode of the rheometer. The average coefficient of friction over the tested frequencies for each of the compounds is tabulated in Table 3. Dulbecco's Phosphate Buffered Saline (DPBS), 2K PEG acid, and 5K PEG acid were used as controls to compare to the synthesized lubricants. Both of the lubricants containing the PAMAM G2 dendrimer did not exhibit coefficient of friction values much different than that of PBS. For example, the coefficient of friction for the 2K PEG-PAMAM G2 dendrimer was 0.1341 and that for DPBS was 0.1376. One-way ANOVA testing among the three (significance level=0.05) produces a p-value of 0.686. Such results indicate that there is no statistical evidence to suggest that there is any difference in the coefficient of friction between DPBS and the 2K PEG-PAMAM G2 and 5K PEG-PAMAM G2 lubricants. In contrast, those lubricants containing PAMAM G3 resulted in significantly lower values: 0.0404 for 2K PEG-PAMAM G3, 0.0319 for 5K PEG-PAMAM G3, and 0.0692 for 20K PEG-PAMAM G3. A paired Student's t-test between coefficient of friction values for DPBS and the 5K PEG-PAMAM G3 lubricant at a significance level of 0.05 yields a p-value of 3.63×10⁻⁷, revealing that there is a strong statistical difference between the two. In addition, there is no statistical difference among the three lubricants containing the PAMAM G3 dendrimer: one-way ANOVA testing between the three at a significance level of 0.05 results in a high p-value of 0.417.

TABLE 3
Coefficient of friction values calculated from the data obtained from the
rheological testing for aluminum-on-steel contact. The coefficient of
friction decreases as the dendrimer generation increases.
Rheological Data for 20% Solutions in DPBS

| Sample Name | Coefficient of Friction |
|---|---|
| DPBS | 0.1376 |
| 2K PEG Acid | 0.0977 |
| 5K PEG Acid | 0.1056 |
| 2K PEG-PAMAM G2 | 0.1341 |
| 5K PEG-PAMAM G2 | 0.1205 |
| 2K PEG-PAMAM G3 | 0.0404 |
| 5K PEG-PAMAM G3 | 0.0319 |
| 20K PEG-PAMAM G3 | 0.0692 |

In addition, measurements were taken with 10% samples in DPBS; however, only data could be collected for the 5K PEG-PAMAM G3 lubricant, which exhibited a coefficient of friction of 0.0385. This value is close to that of a 20% 5K PEG-PAMAM G3 solution. Data could not be obtained for the other compounds at 10% because they were not exhibiting any lubricating properties. The rheometer stops collecting data if the upper plate cannot spin once frictional forces become too high. Moreover, solutions greater than 20%, such as 30 and 40%, could not be made because low reaction yields did not allow for creation of such higher concentrations in 1 mL of DPBS.

### Example 14

Cartilage-on-Cartilage Rheology. A method for simulating a cartilage-on-cartilage contact environment for rheological testing was investigated. The AR 1000 controlled strain rheometer was modified with adapter plugs was customed designed (FIG. 8) to contain opposing conformational cartilage surfaces taken from the femur and tibia of bovine knee joints. The design consisted of an adapter plate modified to connect to another piece by a screw thread (FIG. 8a) to cover and lock around the steel plate of the rheometer. A bottom adapter piece (FIG. 8b) holds the cartilage plug from the tibia to screw into the adapter plate. Additionally, a top adapter plug (FIG. 8c) screws into the top of the rheometer and secures the cartilage plug extracted from the bovine femur.

Cartilage plugs were extracted from the bovine bones using a coring bit to section out a 20 mm long sample with a diameter of 8 mm from both the femur and the tibia. To prevent any bending or compression of the samples, a horizontal cut through the bone was made 20 mm below the top surface. After the cut was made, the cylindrical plug piece fell out and was ready for testing. Conformational surfaces were chosen for uniformity. Plugs from the femur were taken from the center of the bone where there is a small radius of curvature, while the corresponding pieces from the tibia were taken from the flat surfaced center. The plugs fit snuggly into the top and bottom adapter pieces, but were surrounded by a polymethylmethacrylate (PMMA) glue to ensure they would stay in place.

FIG. 8 shows the experimental setup once the lubricant and cartilage plugs were put into place. Testing for cartilage-on-cartilage contact was performed following the same procedure outlined for aluminum-on-steel contact. In order to determine the effectiveness of the experimental setup, four different compounds with known friction-reducing capabilities were tested: phosphate buffered saline (PBS), Synvisc®, hyaluronic acid (HA), and an oxa-norbornene compound synthesized in the Grinstaff lab (FB). The PEGylated dendrimer compounds were not tested because its friction reducing properties in an ex-vivo setting were unknown. The purpose of this experiment was to determine the effectiveness of the cartilage plug procedure. All of the compounds exhibit a coefficient of friction that becomes closer to 0.01 as the angular velocity increases. However, every compound should not exhibit the same behavior, especially PBS. These initial tests therefore indicate that the cartilage used for the plugs was too intact to obtain a noticeable difference between the lubricants. Cartilage can be appropriately degraded using an agent, such as guanidinium chloride, to degrade the collagen fibers. By degrading the cartilage to simulate that of an osteoarthritic patient, one will be able to obtain better results and see an apparent difference between the lubricants, including how effective each would be in decreasing frictional forces in degraded synovial joints.

It is expected that biolubricants according to the present invention will demonstrate properties sufficiently comparable to or better than those of synovial fluid when so tested.

### Example 15

Injection of polymers. New Zealand White Rabbit elbow joints are used to perform lubricant injections. The dendritic polymers are mixed with an iodinated contrast agent (10% by volume) and stirred to ascertain a uniform mixture. The synovial joint space is accessed using a 22G needle under fluoroscopic guidance (OEC 6600). The native synovial fluid is removed, then the mixture (containing the polymer and contrast agent) is injected into the joint space. Delivery of the mixture into the joint space and spacing of the joints thereafter are observed by X-ray analysis.

### Example 16

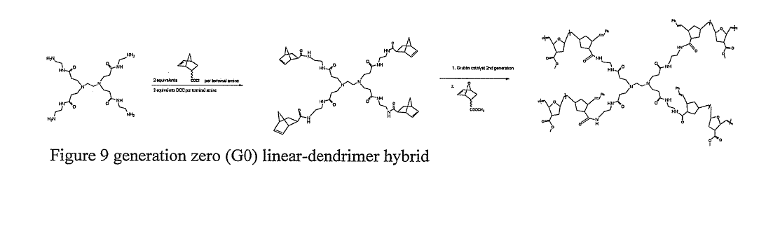

Linear-Dendrimer Hybrids. The terminal amine groups of generation coupling generation zero (G0), generation 1 (G1), generation 2 (G2) and generation 3 (G3) PAMAM dendrimers are coupled to 5-norbornene-2-acyl chloride with the use of a coupling agent, such as N-dicyclohexylcarbodiimide (DCC), to afford PAMAM dendrimers with terminal norbenyl groups. These norbenyl units are exposed to Grubbs catalyst 2^{nd} generation and thus serve as polymer initiation points. Standard ring-opening metathesis polymerization (ROMP) conditions are employed to grow poly (5-oxanorbornene-2methylester) at a determined monomer to catalyst ratio (FIG. 9). These linear-dendrimer hybrids are characterized by size exclusion chromatography (SEC), matrix-assisted laser desorption/ionization (MALDI) and rheology.

### Example 17

Linear-Dendrimer Hybrids. Two hybrid linear-dendritic polymers are prepared that contained a PEG core and a lysine dendron. The structures can possess PEGs of various molecular weight from 1000 to 20,000 g/mol. Likewise the lysine dendron can be a generation 1, 2, 3 or higher. For the representative examples shown here, 20,000 MW poly(ethylene glycol), 1-ethyl-3-(3-dimethylaminopropyl)carbodiimide hydrochloride, hydroxybenzotriazole, and Boc-lys(Boc)-CO₂H are reacted in methanol. Alternatively, the reaction is run with (Boc-lys(Boc)-)₂Lys-CO₂H. The reaction can also be run with a PEG functionalized with two terminal amines and thus yield an amide after the coupling reaction instead of an ester. The reaction is left to run for 24 hours before purification with a molecular weight cutoff spin concentrator tube. The products are characterized and confirmed by NMR and Mass Spectroscopy.

### Example 18

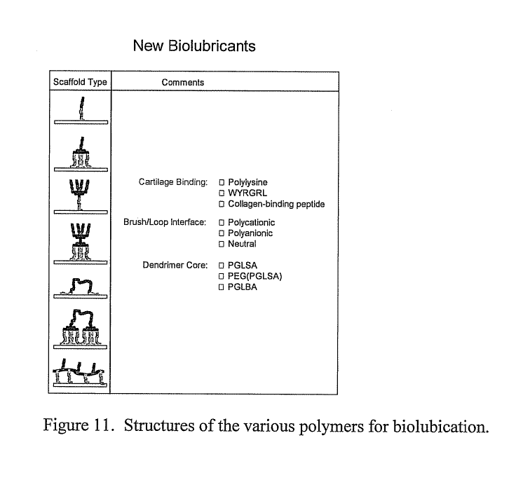

Using the concepts described in this application a number of polymer structures can be prepared. A graphical representation of these structures is shown in FIG. 11.

### Example 19

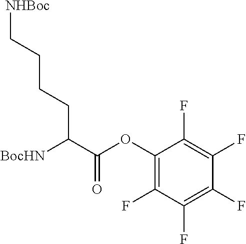

Formation of Boc-Lys(Boc)-OPFP. Dicyclohexylammonium bis-Boc-protected lysine carboxylate (Boc-Lys(Boc)-OH.DHCA), 3.0 g, 5.7 mmol) was dissolved in CH₂Cl₂ and the solution was washed with three times with 0.1 N HCl to remove the DHCA. The organic layer was dried over Na₂SO₄ before starting the reaction. Pentafluorophenol (PFP, 1.2 g, 6.3 mmol) was added to the solution. Dicyclohexylcarbodiimide (DCC, 1.3 g, 6.3 mmol) dissolved in CH₂Cl₂ was added dropwise to the solution over 5 min. The reaction was stirred under N₂ atmosphere at room temperature for 3 h. After this time, the white solid urea byproduct was removed by filtration. Hexanes were added to the filtrate and the flask was placed at −20° C. to induce crystallization of the desired PFP ester. The product was isolated as a white solid in 93% yield.

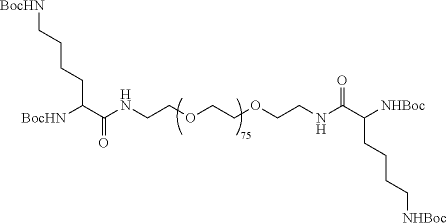

Formation of BocLys-PEG3400-BocLys. 3400 molecular weight diaminopoly(ethylene glycol) (0.3 g, 0.088 mmol) was dissolved in 5 mL of freshly distilled CH₂Cl₂ along with diisopropylethylamine (DIPEA, 0.077 mL, 0.44 mmol). Boc-Lys(Boc)-OPFP (0.18 g, 0.35 mmol) was added to the stirring solution as a solid and the reaction was allowed to proceed under N₂ atmosphere at room temperature for 15 h. After this time, the reaction was added dropwise to cold diethyl ether (−20° C.) to precipitate the desired product. The white solid was isolated by filtration and dried under vacuum. The product was isolated in a 65% yield.

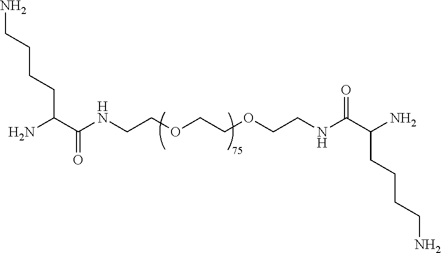

Formation of Lys-PEG3400-Lys. The BocLys-PEG3400-BocLys (0.5 g, 0.12 mmol) species was dissolved in 6 mL of 1:1 CH₂Cl₂/trifluoroacetic acid (TFA) and stirred under N₂ atmosphere at room temperature for 2 h. After this time, the solvent was removed by rotary evaporation and the product was dried under vacuum. The desired product was isolated as a transparent film in quantitative yield.

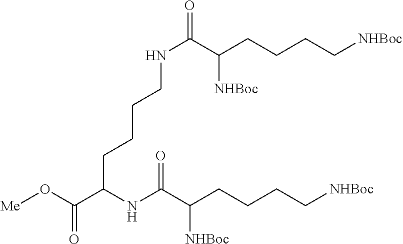

Formation of BocLys3-OMe. Lysine methyl ester (0.36 g, 1.5 mmol) and diisopropylethylamine (1.17 mL, 6.7 mmol) were dissolved in 10 mL of CH₂Cl₂. Boc-Lys(Boc)-OPFP (1.5 g, 2.9 mmol) was added to the solution as a solid. The solution was stirred at room temperature under a N₂ atmosphere for 15 h. After this time, the reaction was concentrated by rotary evaporation and purified by silica gel chromatography (98:2 CH₂Cl₂/MeOH). After pooling the pure fractions and concentrating by rotary evaporation, the product was isolated as a white solid in 91% yield.

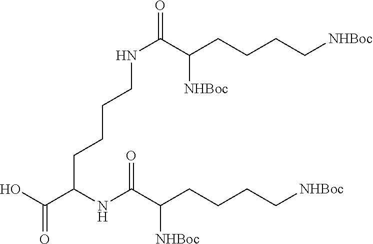

Formation of BocLys3-OH. BocLys3-OMe (1.0 g, 1.2 mmol) was dissolved in 20 mL of 1:1 THF/1N NaOH. The reaction was allowed to stir at room temperature under a N₂ atmosphere for 15 h. After this time, the reaction was concentrated by rotary evaporation to remove the THF. The pH of the remaining aqueous solution was lowered to ˜3.0 by dropwise addition of 1N HCl, then extracted with three 30 mL aliquots of CH₂Cl₂. The organic layers were combined and dried over Na₂SO₄ before being concentrated by rotary evaporation. The product was isolated as a white solid in quantitative yield.

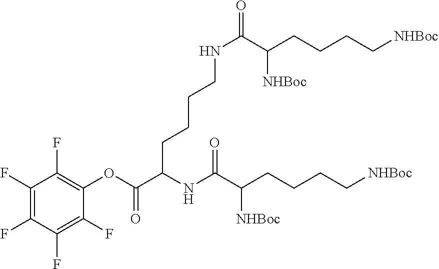

Formation of BocLys3-OPFP. BocLys3-OH (0.9 g, 1.1 mmol) was dissolved in 6 mL of CH₂Cl₂ along with pentafluorophenol (0.23 g, 1.2 mmol). Dicyclohexylurea (0.25 g, 1.2 mmol) was dissolved in CH₂Cl₂ and added to the reaction dropwise. The reaction was stirred at room temperature under a N₂ atmosphere for 15 h. After this time, the white solid urea byproduct was removed by filtration through celite. The desired product was taken on without further purification.

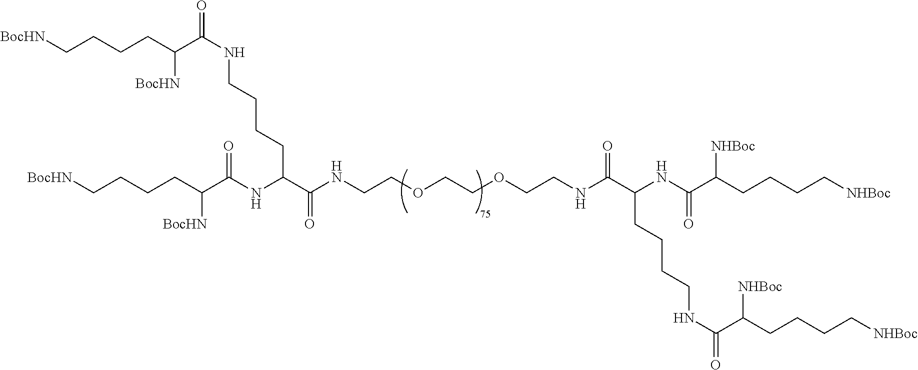

Formation of BocLys3-PEG3400-BocLys3. 3400 molecular weight diaminopoly(ethylene glycol) (0.3 g, 0.088 mmol) was dissolved in 10 mL of freshly distilled CH₂Cl₂ along with diisopropylethylamine (DIPEA, 0.077 mL, 0.44 mmol). BocLys3-OPFP (0.34 g, 0.35 mmol) was added to the stirring solution as a solid and the reaction was allowed to proceed under N₂ atmosphere at room temperature for 15 h. After this time, the reaction was added dropwise to cold diethyl ether (−20° C.) to precipitate the desired product. The white solid was isolated by filtration and dried under vacuum. The product was isolated in a 65% yield.

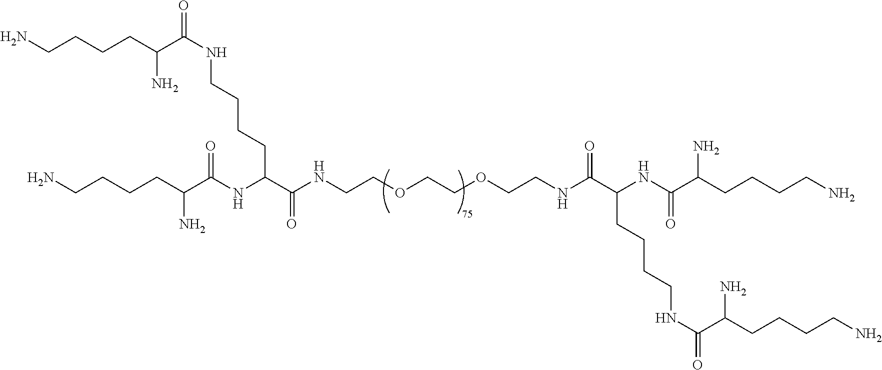

Formation of Lys3-PEG3400-Lys3. The BocLys3-PEG3400-BocLys3 (0.06 g, 0.012 mmol) species was dissolved in 6 mL of 1:1 CH₂Cl₂/trifluoroacetic acid (TFA) and stirred under N₂ atmosphere at room temperature for 2 h. After this time, the solvent was removed by rotary evaporation and the product was dried under vacuum. The desired product was isolated as a transparent film in quantitative yield.

### Other Embodiments

Other embodiments of the invention will be apparent to those skilled in the art from a consideration of the specification or practice of the invention disclosed herein. It is intended that the specification and Examples be considered as exemplary only, with the true scope of the invention being indicated by the following claims.

What is claimed is:

1. A branched polymer having a molecular weight of greater than 5,000 g/mol for use as a lubricant or shock absorber in vivo.

2. The polymer of claim 1, wherein the polymer is in the form of a viscous liquid.

3. The polymer of claim 1, wherein the polymer is in the form of a gel.

4. The polymer of claim 1, wherein the polymer is not crosslinkable.

5. The polymer of claim 1, wherein the polymer is crosslinkable.

6. The polymer of claim 1, wherein the polymer is a dendrimer.

7. The polymer of claim 1, wherein the polymer is a hybrid linear-dendrimer.

8. The polymer of claim 1, wherein the polymer is a hyperbranched polymer.

9. The polymer of claim 1, wherein the polymer has one of the following general formulas: 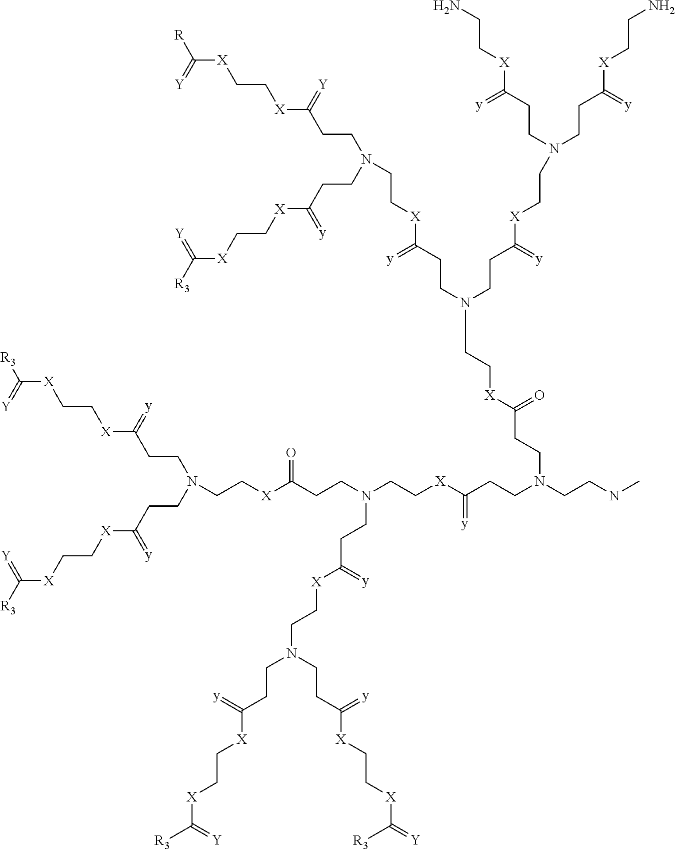 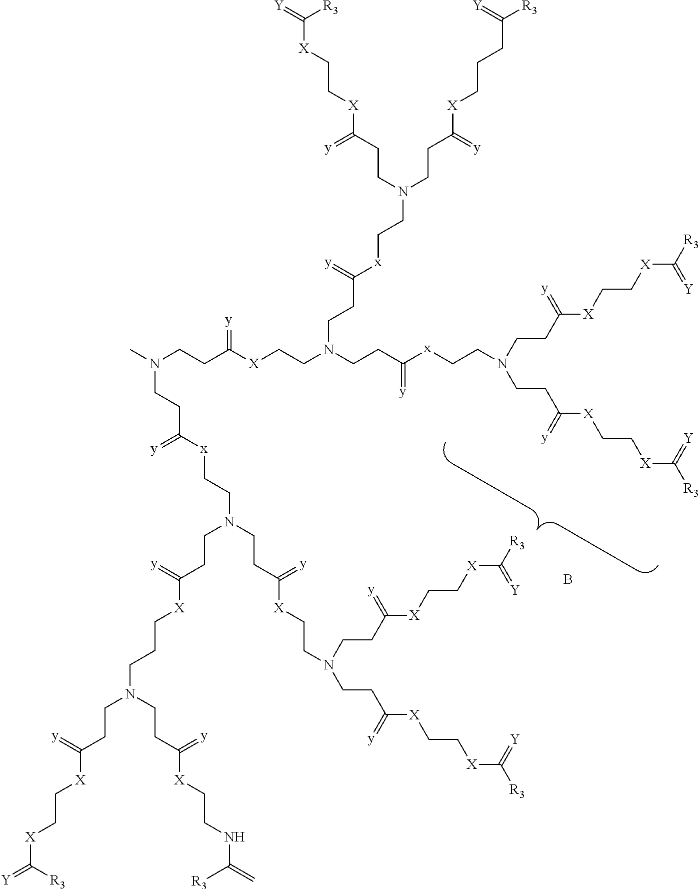 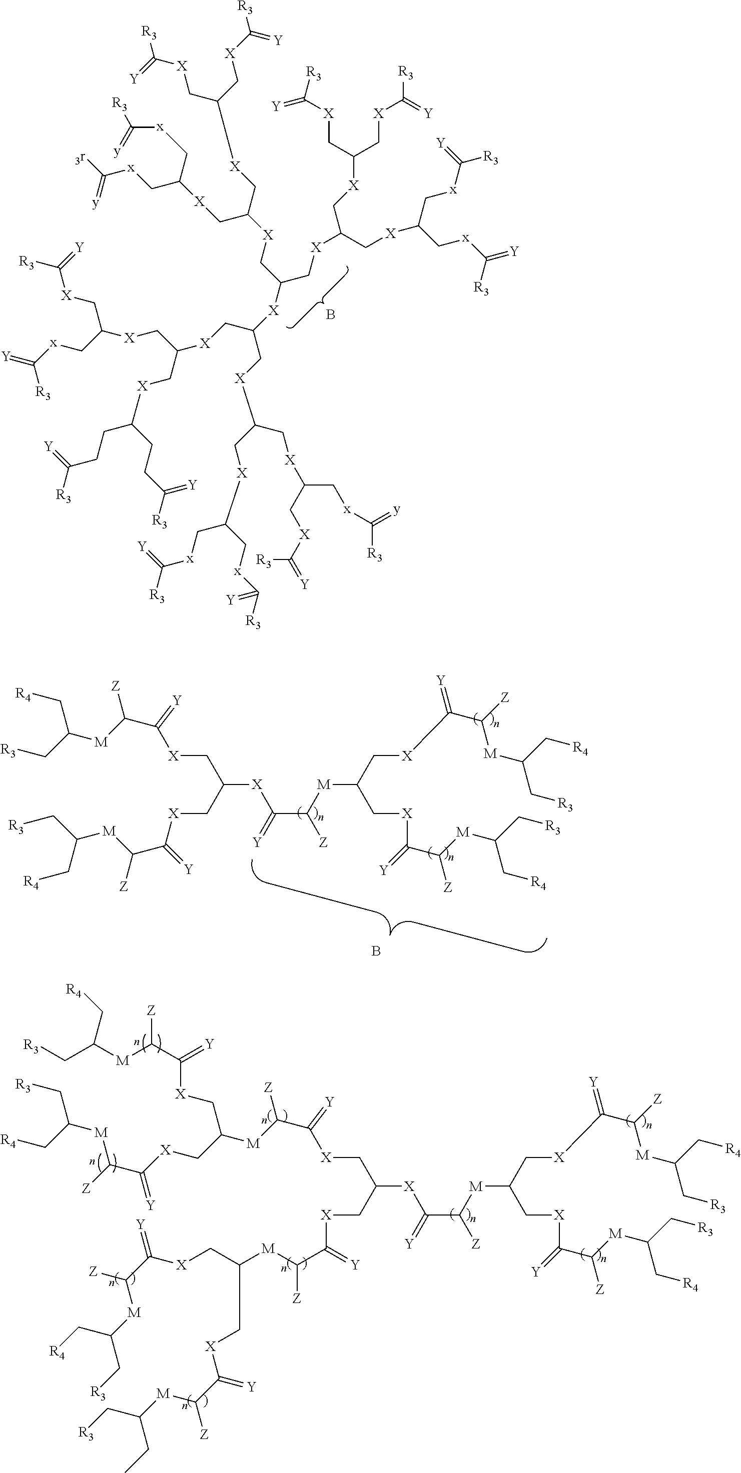 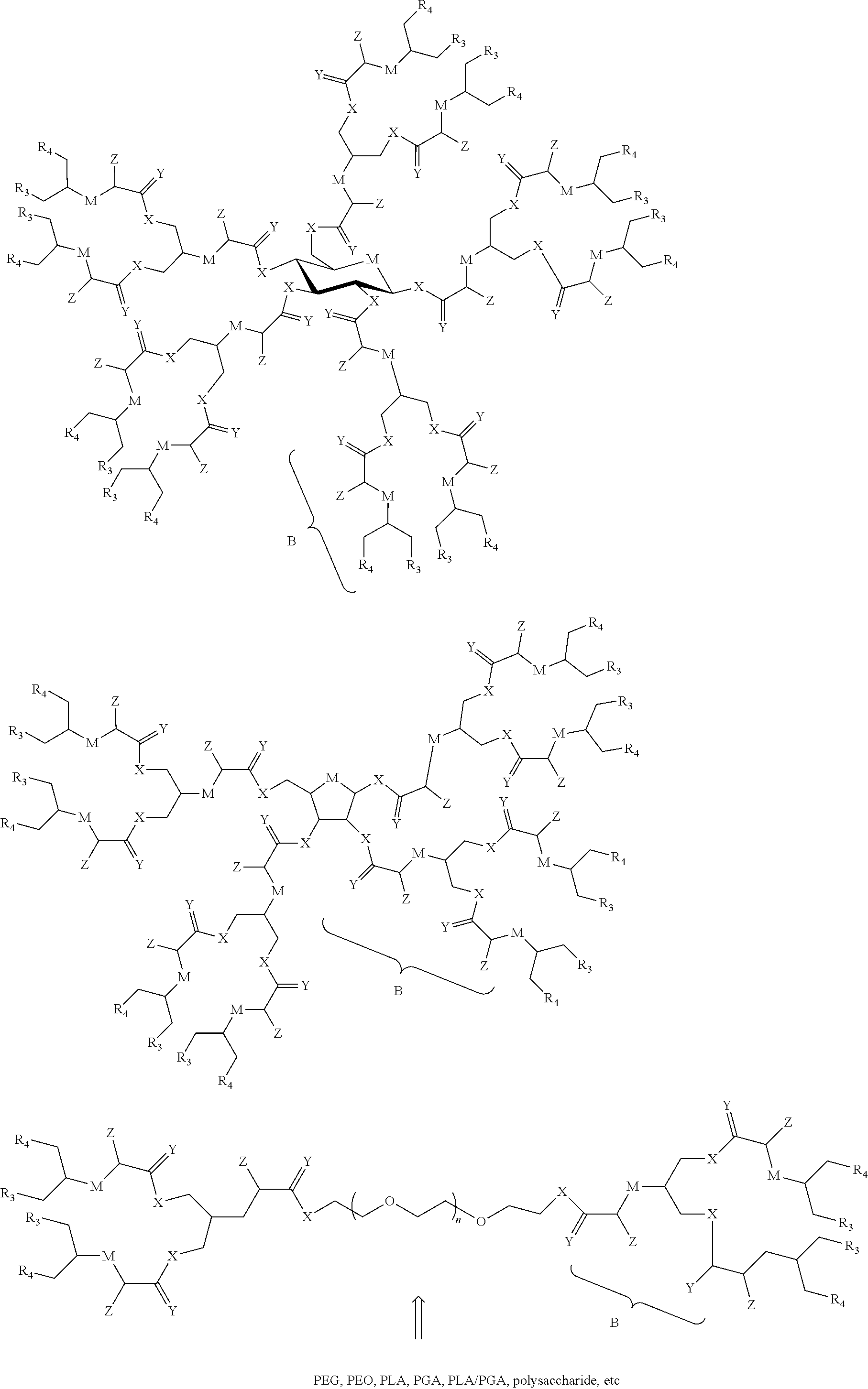 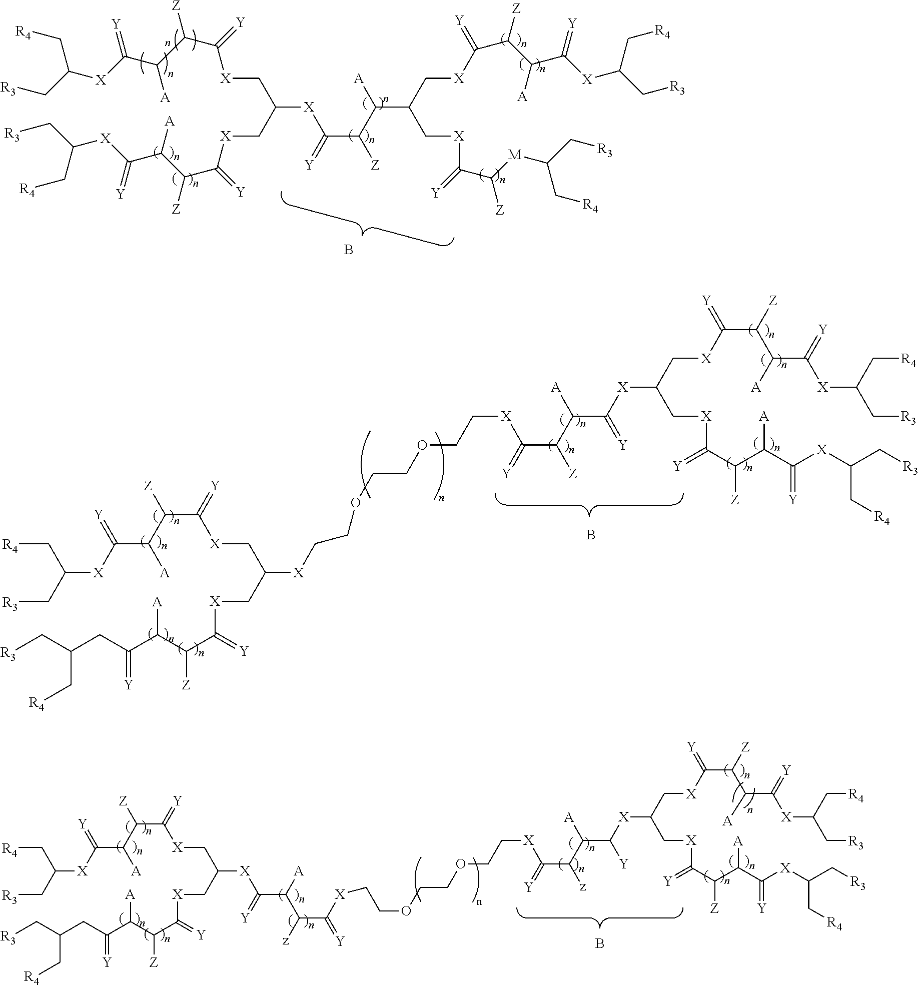 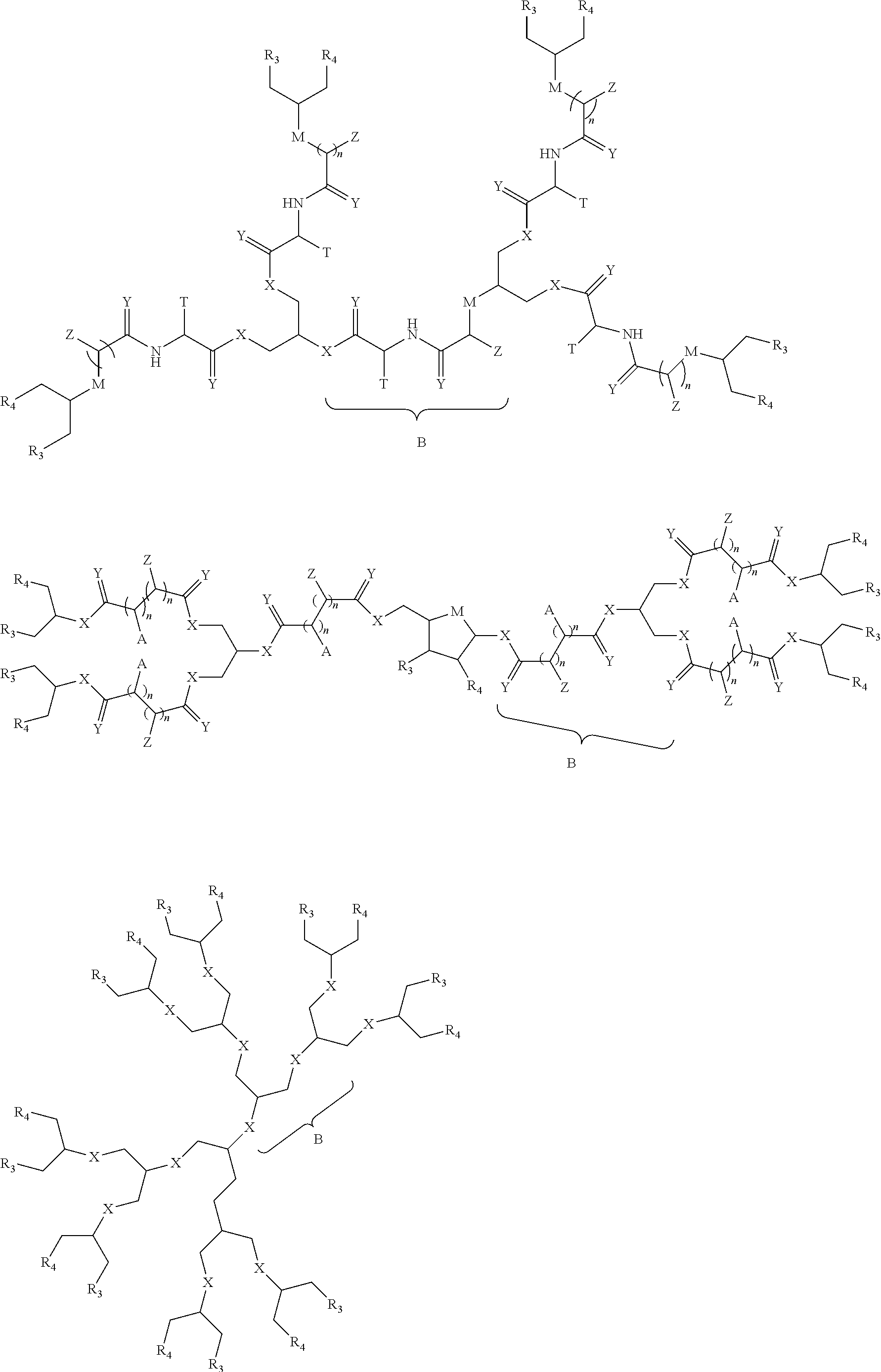 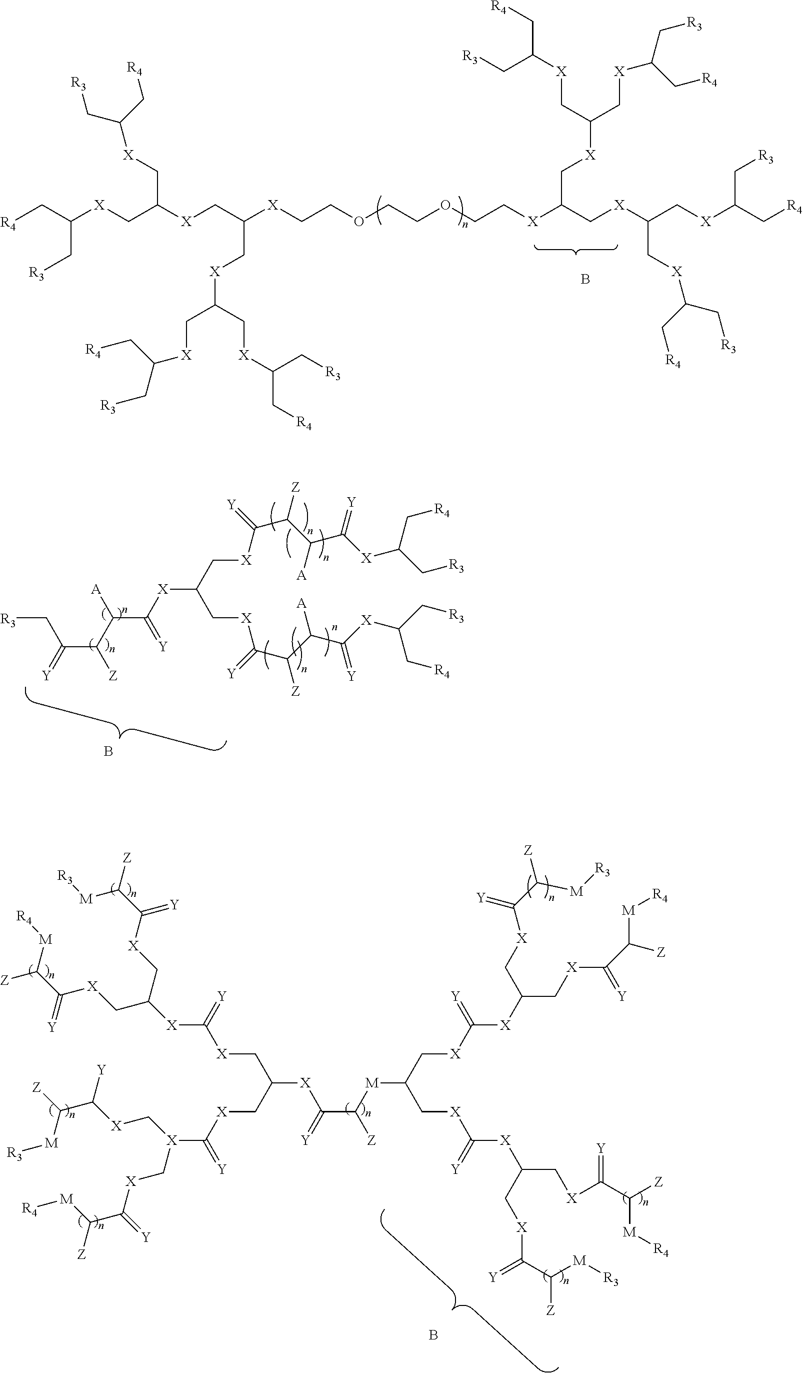
wherein:
n is an integer independently selected from 0 to 50, inclusive;
c is a natural or un-natural amino acid;
each occurrence of R₃, R₄, A, and Z is independently selected from the group consisting of a repeat pattern of B, an optionally substituted C₁₋₅₀ aliphatic group, —H, —OH, —CH₃, carboxylic acid, sulfate, phosphate, aldehyde, methoxy, amine, amide, thiol, disulfide, straight or branched chain alkane, straight or branched chain alkene, straight or branched chain ester, straight or branched chain ether, straight or branched chain silane, straight or branched chain urethane, straight or branched chain carbonate, straight or branched chain sulfate, straight or branched chain phosphate, straight or branched chain thiol urethane, straight or branched chain amine, straight or branched chain thiol urea, straight or branched chain thiol ether, straight or branched chain thiol ester, a carboxylic acid protecting group, and a linker moiety; and
each occurrence of X, Y, and M is independently selected from the group consisting of O, S, Se or any other isoelectronic species of oxygen; and or N(R′)_{n′}, wherein R′ is hydrogen or an optionally substituted C₁₋₂₀ aliphatic group or an optionally substituted aromatic group; and wherein n′ is an integer from 1-4, inclusive.

10.-25. (canceled)

26. The polymer of claim 9, wherein R₃ is a carboxylic acid protecting group.

27. The polymer of claim 9, wherein R₃ is a phthalimidomethyl ester, a t-butyldimethylsilyl ester, or a t-butyldiphenylsilyl ester.

28. The polymer of claim 9, wherein the polymer includes a straight or branched chain of 1-50 carbon atoms.

29. The polymer of claim 28, wherein the straight or branched chain is fully saturated.

30. The polymer of claim 28, wherein the straight or branched chain is fully unsaturated.

31. The polymer of claim 28, wherein the straight or branched chain is partially saturated.

32. The polymer of claim 9, wherein R₃, R₄, A, and Z are any combination of linkers selected from the group consisting of esters, silanes, ureas, amides, amines, urethanes, thiol-urethanes, carbonates, thio-ethers, thio-esters, sulfates, phosphates and ethers.

33. The polymer of claim 9, wherein the straight or branched chain includes at least one group consisting of flourocarbons, halocarbons, alkenes, and alkynes.

34. The polymer of claim 9, wherein the straight or branched chain includes one or more photopolymerizable groups and a polyether, polyester, polyamine, polyacrylic acid, polyamino acid, polynucleic acid or polysaccharide with a molecular weight in the range from 5000 to 10,000,000 g/mol.

35. The polymer of claim 9, wherein the straight or branched chain includes at least one PPG, PEG, PLA, PGA, PGLA, or PMMA polymer with a molecular weight in the range of 500 to 50,000 g/mol.

36. The polymer of claim 9, wherein the polymer includes at least one terminal group selected from the group consisting of amines, thiols, amides, phosphates, sulphates, hydroxides, alkenes, and alkynes.

37. The polymer of claim 9, wherein a molecule is attached to at least one Z, A, R₃, and/or R₄ group and the molecule is selected from the group consisting of a polypeptide, an antibody, a nucleotide, a nucleoside, an oligonucleotide, a ligand, a pharmaceutical agent or a carbohydrate.

38.-45. (canceled)

46. The polymer of claim 37, wherein the molecule is a carbohydrate and is mannose or sialic acid.

47. The polymer of claim 9, wherein a PET or MRI contrast agent is attached to at least one Z, A, R₃, and/or R₄ group.

48. The polymer of claim 47, wherein the contrast agent is Gd(DPTA).

49. The polymer of claim 9, wherein an iodated compound for X-ray imaging is attached to at least one Z, A, R₃, and/or R₄ group.

50. The polymer of claim 9, wherein a pharmaceutical agent is attached to at least one Z, A, R₃, and/or R₄ group, wherein the pharmaceutical agent is selected from the group consisting of antibacterial, anticancer, anti-inflammatory, and antiviral agents.

51. The polymer of claim 1, wherein the polymer contains at least one stereochemical center.

52. The polymer of claim 51, wherein the at least one stereochemical center is chiral.

53. The polymer of claim 51, wherein the at least one stereochemical center is achiral.

54. The polymer of claim 1, wherein the polymer contains at least one site where the branching is incomplete.

55. The polymer of claim 1, wherein the polymer was made by a convergent synthesis.

56. The polymer of claim 1, wherein the polymer was made by a divergent synthesis.

57. The polymer of claim 1, wherein the polymer was made by a combination of a divergent and convergent synthesis.

58. A pharmaceutical composition for use as a lubricant or shock absorber in vivo, wherein the pharmaceutical composition comprises an effective amount of at least one polymer of claim 1 and at least one pharmaceutically acceptable carrier.

59. The pharmaceutical composition of claim 58 further comprising a bioactive agent.

60. The pharmaceutical composition of claim 59, wherein the bioactive agent is selected from the group consisting of a growth factor, a cytokine, a small molecule, an analgesic, an anesthetic, an antimicrobial agent, an antibacterial agent, an antiviral agent, an antifungal agent, an antibiotic, an anti-inflammatory agent, an antioxidant, an antiseptic agent, and any combination thereof.

61. The pharmaceutical composition of claim 59, wherein the bioactive agent is selected from the group consisting of collagen, fat, silicone paste, poly(tetrafluoroethylene) paste, calcium hydroxyapatite, hyaluronic acid, hyaluronates, and any combination thereof.

62. A method comprising administering an effective amount of a polymer of claim 1 to a subject in need thereof.

63.-121. (canceled)
